(19) United States
(12) Patent Application Publication
(10) Pub. No.: US 2008/0241069 A1
(43) Pub. Date: Oct. 2, 2008

(54) Methods and compositions for treating IgE-mediated diseases

(76) Inventor: Yvonne Paterson, Philadelphia, PA (US)

Correspondence Address: Pearl Cohen Zedek Latzer, LLP, 1500 Broadway, 12th Floor, New York, NY, 10036, US

(21) Appl. No.: 11/882,782

(22) Filed: Aug. 6, 2007

Related U.S. Application Data
(60) Provisional application No. 60/835,420, filed on Aug. 4, 2006.

(51) Int. Cl. A61K 49/00; C12P 21/00; A61K 39/395; C12N 1/21; C12N 15/64; C07H 21/00; A61K 39/00; A61K 39/02; A61P 11/06; A61P 37/04

(52) U.S. Cl. 424/9.2; 530/387.3; 424/134.1; 435/252.3; 536/23.5; 424/184.1; 424/200.1; 435/320.1

(57) ABSTRACT

This invention provides recombinant peptides comprising a fragment of an IgE constant region, nucleotide molecules encoding same, recombinant vaccine vectors comprising same, and methods for inducing immune response and treating allergy, asthma and IgE mediated disease comprising same.

### CROSS REFERENCE TO RELATED APPLICATIONS

This application claims priority from U.S. Provisional Application Ser. No. 60/835,420 filed Aug. 4, 2006, which is incorporated in its entirety herein by reference.

### FIELD OF INVENTION

This invention provides recombinant peptides comprising a fragment of an IgE constant region, nucleotide molecules encoding same, recombinant vaccine vectors comprising same, and methods for inducing immune response and treating allergy and asthma, comprising same.

### BACKGROUND OF THE INVENTION

Asthma is clinically characterized by one or more of episodic airflow obstruction, inflammation of the airways, and enhanced bronchial reactivity (airway hyper-reactivity [AHR]) to inhaled spasmogenic stimuli. The mechanisms underlying the development of AHR and diminished airflow are considered to play central roles in disease pathogenesis. Although the etiology of asthma is complex, inflammation of the airways, elicited by an inappropriate immune response to inhaled allergens, is considered a principle predisposing factor for the clinical expression and pathogenesis of this disorder. Disease severity often correlates with progressive inflammation of the airways as well as the levels of airways obstruction and AHR.

CD4⁺Th2 lymphocytes (Th2 cells) are predominant features of inflammatory infiltrates in asthma. These cells are thought to regulate disease progression and AHR by secreting cytokines that induce the immune and pathologic responses (e.g. IgE production) that can be features of this disease. Methods for treating and ameliorating asthma and allergy are urgently needed in the art.

### SUMMARY OF THE INVENTION

This invention provides recombinant peptides comprising a fragment of an IgE constant, region, nucleotide molecules encoding same, recombinant vaccine vectors comprising same, and methods for inducing immune response and treating allergy and asthma, comprising same.

In one embodiment, the present invention provides a recombinant peptide comprising a fragment of an IgE constant region, and a non-IgE amino acid (AA) sequence. In another embodiment, the non-IgE AA sequence is a listeriolysin (LLO) AA sequence. In another embodiment, the non-IgE AA sequence is an ActA AA sequence. In another embodiment, the non-IgE AA sequence is a PEST-like AA sequence. In another embodiment, the non-IgE AA sequence is any other non-IgE AA sequence known in the art. Each possibility represents a separate embodiment of the present invention.

In another embodiment, the present invention provides a vaccine comprising a recombinant polypeptide of the present invention.

In another embodiment, the present invention provides an immunogenic composition comprising a recombinant polypeptide of the present invention.

In another embodiment, the present invention provides a recombinant vaccine vector encoding a recombinant polypeptide of the present invention.

In another embodiment, the present invention provides a recombinant Listeria strain comprising a recombinant polypeptide of the present invention.

In another embodiment, the present invention provides a method of inducing a cell-mediated immune response against an IgE protein in a subject, the method comprising contacting the subject with an immunogenic composition comprising either (a) a recombinant peptide comprising the IgE protein or a fragment thereof; or (b) a nucleotide molecule encoding the recombinant peptide, thereby inducing a cell-mediated immune response against an IgE protein in a subject. In another embodiment, the cell-mediated immune response is a T cell response. In another embodiment, the IgE protein is endogenously expressed within the subject. Each possibility represents a separate embodiment of the present invention.

In another embodiment, the present invention provides a method of treating, inhibiting, suppressing or ameliorating an allergy-induced asthma in a subject, comprising the step of contacting the subject with an immunogenic composition comprising either (a) a recombinant peptide comprising an IgE protein or a fragment thereof; or (b) a nucleotide molecule encoding the recombinant peptide, thereby treating, inhibiting, suppressing or ameliorating an allergy-induced asthma in a subject. In another embodiment, the IgE protein is endogenously expressed by the subject. Each possibility represents a separate embodiment of the present invention.

In another embodiment, the present invention provides a method of treating, inhibiting, suppressing or ameliorating an allergy in a subject, comprising the step of contacting the subject with an immunogenic composition comprising either (a) a recombinant peptide comprising an IgE protein or a fragment thereof; or (b) a nucleotide molecule encoding the recombinant peptide, thereby treating, inhibiting, suppressing or ameliorating an allergy in a subject. In another embodiment, the IgE protein is endogenously expressed by the subject. Each possibility represents a separate embodiment of the present invention.

In another embodiment, the present invention provides a method of reducing an incidence of an asthma episode in a subject, comprising the step of contacting the subject with an immunogenic composition comprising either (a) a recombinant peptide comprising an IgE protein or a fragment thereof; or (b) a nucleotide molecule encoding the recombinant peptide, wherein the IgE protein is endogenously expressed by a cell of the subject, and wherein the immunogenic composition induces a formation of a T cell-mediated immune response against the IgE protein, thereby reducing an incidence of an asthma episode in a subject. In another embodiment, the recombinant peptide further comprises a non-IgE AA sequence. In another embodiment, the non-IgE AA sequence is any non-IgE AA sequence enumerated herein. Each possibility represents a separate embodiment of the present invention.

In another embodiment, the present invention provides a method of treating, inhibiting, suppressing, or ameliorating an IgE-mediated disease or disorder in a subject, comprising the step of contacting said subject with an immunogenic composition comprising either (a) a recombinant peptide comprising an IgE protein or a fragment thereof; or (b) a nucleotide molecule encoding said recombinant peptide, wherein said IgE protein is endogenously expressed by a cell of said subject, and wherein said immunogenic composition induces a formation of a T cell-mediated immune response against said IgE protein, thereby treating, inhibiting, suppressing, or ameliorating an IgE-mediated disease or disorder in a subject. In one embodiment, the IgE-mediate disease or disorder comprises asthma, allergy-induced asthma, hay fever, drug allergies, pemphigus vulgaris, atopic dermatitis, urticaria, eczema conjunctivitis, rhinorrhea, rhinitis gastroenteritis, myeloma, Hodgkin's disease, Hyper-IgE syndrome, Wiskott-Aldrich syndrome, or a combination thereof. Each possibility represents a separate embodiment of the present invention.

In another embodiment, the present invention provides a method of identifying a compound that ameliorates an IgE-mediated disease or disorder, the method comprising the steps of: (a) contacting a first animal with said compound, wherein said first animal has not been administered the recombinant peptide of claim 1 and wherein said first animal exhibits said IgE-mediated disease or disorder; (b) contacting a second animal with said compound, wherein said first animal has been administered the recombinant peptide of claim 1; and (c) measuring a clinical correlate of said IgE-mediated disease or disorder in said first animal and said second animal; whereby, if said compound positively affects said clinical correlate in said first animal and does not affect said clinical correlate in said second animal, then said compound ameliorates said IgE-mediated disease or disorder.

### BRIEF DESCRIPTION OF THE FIGURES

FIG. 1. Lm-E7 vs. Lm-LLO-E7. Lm-E7 was generated by introducing a gene cassette into the orfz domain of the Listeria monocytogenes (LM) genome (A). The hly promoter drives expression of the hly signal sequence and the first five amino acids (AA) of LLO followed by HPV-16 E7. B), Lm-LLO-E7 was generated by transforming the prfA-strain XFL-7 with the plasmid pGG-55. pGG-55 has the hly promoter driving expression of a nonhemolytic fusion of LLO-E7 and the prfA gene to select for retention of the plasmid.

FIG. 2. Lm-E7 and Lm-LLO-E7 secrete E7. Lm-Gag (lane 1), Lm-E7 (lane 2), Lm-LLO-NP (lane 3), Lm-LLO-E7 (lane 4), XFL-7 (lane 5), and 10403S (lane 6) were grown overnight at 37° C. in Luria-Bertoni broth. Equivalent numbers of bacteria, as determined by OD at 600 nm absorbance, were pelleted and 18 ml of each supernatant was TCA precipitated. E7 expression was analyzed by Western blot. The blot was probed with an anti-E7 mAb, followed by HRP-conjugated anti-mouse (Amersham), then developed using ECL detection reagents.

FIG. 3. Schematic representation of the pActA-E7 expression system used to express and secrete E7 under hly promoter (pHLY) from recombinant Listeria strains. The prfA gene was used to select retention of the plasmid.

FIG. 4. (A) Western blot demonstrating that Lm-ActA-E7 secretes ActA-E7, (about 64 kD). Gels were transferred to polyvinylidene difluoride membranes and probed with 1:2500 anti-E7 monoclonal antibody, then with 1:5000 horseradish peroxidase-conjugated anti-mouse IgG. Lane 1: Lm-LLO-E7; lane 2: Lm-ActA-E7.001; lane 3; Lm-ActA-E7-2.5.3; lane 4: Lm-ActA-E7-2.5.4. (B) Magnification of a portion of the Western blot from part (A).

FIG. 5. Tumor size in mice immunized with Lm-ActA-E7 (solid rectangles), Lm-LLO-NP (hollow triangles), and naive mice (non-vaccinated; circles) on days 7 and 14 after subcutaneous implantation of TC-1 tumor cells.

FIG. 6. A. Induction of E7 specific IFN-gamma secreting CD8⁺ T cells in the spleens and tumors of mice administered TC-1 tumor cells and subsequently administered Lm-E7, Lm-LLO-E7, Lm-ActA-E7 or no vaccine (naive). B. Induction and penetration of E7 specific CD8⁺ cells in the spleens and tumors of mice administered TC-1 cells and subsequently administered a recombinant Listeria vaccine (naive, Lm-LLO-E7, Lm-E7, Lm-ActA-E7).

FIG. 7. A. Induction of E7-specific CTL by Lm-ActA-E7 vaccination. B. Control experiment using EL4 target cells not expressing E7.

FIG. 8. Listeria constructs containing PEST regions lead to greater tumor regression. A. data from 1 representative experiment. B. average tumor size and SE of data from 3 experiments.

FIG. 9. Listeria constructs containing PEST regions induce a higher percentage of E7-specific lymphocytes in the spleen. A. data from 1 representative experiment. B. average and SE of data from 3 experiments.

FIG. 10. Listeria constructs containing PEST regions induce a higher percentage of E7-specific lymphocytes within the tumor. A. data from 1 representative experiment. B. average and SE of data from 3 experiments.

FIG. 11. Depiction of vaccinia virus constructs expressing different forms of HPV16E7 protein.

FIG. 12. VacLLOE7 induces long-term regression of tumors established from 2×10⁵ TC-1 cells in C57BL/6 mice. Mice were injected 11 and 18 days after tumor challenge with 10⁷ PFU of VacLLOE7, VacSigE7LAMP-1, or VacE7/mouse i.p. or were left untreated (naive). 8 mice per treatment group were used, and the cross section for each tumor (average of 2 measurements) is shown for the indicated days after tumor inoculation.

FIG. 13: FIG. 13. E6/E7 transgenic mice develop tumors in the thyroid, where E7 gene is expressed. Mice were sacrificed at 6 months and thyroids were removed, sectioned, and stained by hematoxylin and eosin. (a) Gross photograph of 18 month old E6/E7 transgenic mouse with enlarged thyroid visible externally. (b) Photomicrograph of a thyroid gland from a 6 month old E6/E7 transgenic mouse. The thyroid follicles are engorged with colloid, and they are irregular in shape. (c) Photomicrograph of a thyroid gland from a 6 month old mouse at higher magnification. Instead of colloid-filled follicles throughout the gland, there exist solid masses of cells with little or no follicular organization. A papillary carcinoma is evident. A normal thyroid at low (d) and high (e) magnification from a 6 month C57BL/6 wild-type mouse is shown for comparison.

FIG. 14. LLO and ActA fusions induce regression of solid tumors in the E6/E7 transgenic mice in wild-type mice and transgenic mice immunized with LM-LLO-E7 (A), or LM-ActA-E7 (B), compared to naïve mice or mice treated with LM-NP (control). Similar experiments were performed with 4 immunizations of LM-LLO-E7 (C), or LM-ActA-E7 (D).

FIG. 15. LM-LLO-E7 and Lm-ActA-E7 vaccines decreased mice thyroid weight. 6 to 8 week old mice were immunized with 1×10⁸ Lm-LLO-E7 or 2.5×10⁸ Lm-ActA-E7 once per month for 8 months. Mice were sacrificed 20 days after the last immunization and their thyroids removed and weighed.

FIG. 16. Lm-LLO-Her-2 vaccines slow the growth of established rat Her-2 expressing tumors in rat Her-2/neu transgenic mice, in which rat Her-2 is expressed as a self-antigen.

FIG. 17. LLO-Her-2 vaccines control spontaneous tumor growth in Her-2/neu transgenic mice.

FIG. 18. In vitro presentation by host cells infected with LM recombinants. J774 cells were infected with bacteria and used as targets in a ⁵¹Cr release assay. Effectors were splenocytes from influenza-immune mice stimulated with the K^{d} restricted NP epitope. Hollow circles: uninfected J774 cells; filled circles: pulsed with the K^{d} restricted NP peptide; hollow squares: infected with strain 10403s; hollow triangles: infected with DP-L2840; filled triangles: infected with DP-L2851; filled squares: infected with DP-L2028.

FIG. 19. Induction of NP-specific CTL after immunization with recombinant LM strains. Splenocytes from mice immunized with DP-L2028 (A) or DP2851 (B) were stimulated in vitro for 5 days with the Kd restricted NP peptide and used as effectors in a ⁵¹Cr release assay. Targets were P815 cells untreated (hollow squares), pulsed with the K^{d} restricted NP peptide (filled squares), pulsed with the K^{d} restricted LLO peptide (filled triangles) or pulsed with the Db restricted NP peptide (filled circles).

FIG. 20. Lung influenza virus titers of lung extracts from mice immunized with the indicated vaccines and in naive mice. Each panel represents an experiment performed on a separate occasion. N=3 for experiments 1 and 2 and 6 for experiments 3 and 4.

### DETAILED DESCRIPTION OF THE INVENTION

This invention provides recombinant peptides comprising a fragment of an IgE constant region, nucleotide molecules encoding same, recombinant vaccine vectors comprising same, and methods for inducing immune response and treating allergy and asthma, comprising same.

In one embodiment, the present invention provides a recombinant peptide comprising a fragment of an IgE constant region (“IgE fragment”), and a non-IgE amino acid (AA) sequence. In another embodiment, the non-IgE AA sequence is a listeriolysin (LLO) AA sequence. In another embodiment, the non-IgE AA sequence is an ActA AA sequence. In another embodiment, the non-IgE AA sequence is a PEST-like AA sequence. As provided herein, fusion to LLO, ActA, PEST-like sequences and fragments thereof enhances the cell-mediated immunogenicity of antigens. In another embodiment, the non-IgE AA sequence is any other immunogenic non-IgE AA sequence known in the art. Each possibility represents a separate embodiment of the present invention.

In one embodiment, a fragment is a portion of a nucleic acid, peptide or protein, which in one embodiment, retains the desired function and/or property of the full nucleic acid, peptide or protein.

An LLO AA sequence of methods and compositions of the present invention is, in another embodiment, a non-hemolytic LLO AA sequence. In another embodiment, the sequence is an LLO fragment. In another embodiment, the sequence is a complete LLO protein. In another embodiment, the sequence is any LLO protein or fragment thereof known in the art. Each possibility represents a separate embodiment of the present invention.

The LLO protein utilized to construct vaccines of the present invention has, in another embodiment, the sequence:

MKKIMLVFITLLVSLPIAQQTEAKDASAFNKENSISSMAPPASPPASPKTPIEKKHADEIDKYIQGLD YNKNNVLVYHGDAVTNVPPRKGYKDGNEYIVVEKKKKSINQNNADIQVVNAISSLTYPGALVKA NSELVENQPDVLPVKRDSLTLSIDLPGMTNQDNKIVVKNATKSNVNNAVNTLVERWNEKYAQAY PNVSAKIDYDDEMAYSESQLIAKFGTAFKAVNNSLNVNFGAISEGKMQEEVISFKQIYYNVNVNEP TRPSRFFGKAVTKEQLQALGVNAENPPAYISSVAYGRQVYLKLSTNSHSTKVKAAFDAAVSGKSV SGDVELTNIIKNSSFKAVIYGGSAKDEVQIIDGNLGDLRDILKKGATFNRETPGVPIAYTTNFLKDNE LAVIKNNSEYETTSKAYTDGKINIDHSGGYVAQFNISWDEVNYDPEGNEIVQHKNWSENNKS KLAHFTSSIYLPGNARNINVYAKECTGLAWEWWRTVIDDRNLPLVKNRNISIWGTTLYPKYSN KVDNPIE (GenBank Accession No. P13128; SEQ ID NO: 1); the nucleic acid sequence is set forth in GenBank Accession No. X15127:
taacgaogataaagggacagcaggactagaataaagctataaagcaagcatataatattgcgtttcatctttagaagcgaatttcgccaatattataatta tcaaaagagaggggtggcaaacggtatttggcattattaggttaaaaaatgtagaaggagagtgaaacccatgaaaaaaataatgctagtttttattacac ttatattagttgtctaccaattgcgcaacaaactgaagcaaaggatgcatctgcattcaataaagaaaattcaatttcatccatggcaccaccagcatctcc gcctgcaagtcctaagacgccaatcgaaaagaaacacgcggatgaaatcgataagtatatacaaggattggattacaataaaaacaatgtattagtata ccacggagatgcagtgacaaatgtgccgccaagaaaaggttacaaagatggaaatgaatatattgttgtggagaaaaagaagaaatccatcaatcaaa ataatgcagacattcaagttgtgaatgcaatttcgagcctaacctatccaggtgctctcgtaaaagcgaattcggaattagtagaaaatcaaccagatgttc tccctgtaaaacgtgattcattaacactcagcattgatttgccaggtatgactaatcaagacaataaaatcgttgtaaaaaatgccactaaatcaaacgttaa caacgcagtaaatacattagtggaaagatggaatgaaaaatatgctcaagcttatccaaatgtaagtgcaaaaattgattatgatgacgaaatggcttaca gtgaatcacaattaattgcgaaatttggtacagcatttaaagctgtaaataatagcttgaatgtaaacttcggcgcaatcagtgaagggaaaatgcaagaa gaagtcattagttttaaacaaatttactataacgtgaatgttaatgaacctacaagaccttccagatttttcggcaaagctgttactaaagagcagttgcaagc gcttggagtgaatgcagaaaatcctcctgcatatatctcaagtgtggcgtatggccgtcaagtttatttgaaattatcaactaattcccatagtactaaagta aaagctgcttttgatgctgccgtaagcggaaaatctgtctcaggtgatgtagaactaacaaatatcatcaaaaattcttccttcaaagccgtaatttacgga ggttccgcaaaagatgaagttcaaatcatcgacggcaacctcggagacttacgcgatattttgaaaaaaggcgctacttttaatcgagaaacaccagga gttcccattgcttatacaacaaacttcctaaaagacaatgaattagctgttattaaaacaactcagaatatattgaaacaacttcaaaagcttataicagatgg aaaaattaacatcgatcactctggaggatacgttgctcaattcaacatttcttgggatgaagtaaattatgatcctgaaggtaacgaaattgttcaacataaa aactggagcgaaaacaataaaagcaagctagctcatttcacatcgtccatctatttgccaggtaacgcgagaaatattaatgtttacgctaaagaatgcac tggtttagcttgggaatggtggagaacggtaattgatgaccggaacttaccacttgtgaaaaatagaaatatctccatctggggcaccacgctttatccga aatatagtaataaagtagataatccaatcgaataattgtaaaagtaataaaaaattaagaataaaaccgcttaacacacacgaaaaaataagcttgttttgca Cctttcgtaaattattttgtgaagaatgtagaaacaggcttattttttaatttttttagaagaattaacaaatgtaaaagaatatctgactgtttatccatataatat aagcatatcccaaagtttaagccacctatagtttctactgcaaaacgtataatttagttccccacatatactaaaaaacgtgtccttaactctctctgtcagatta gttgta (SEQ ID No: 44). The first 25 AA of the proprotein corresponding to this sequence are the signal sequence and are cleaved from LLO when it is secreted by the bacterium. Thus, in this embodiment, the full length active LLO protein is 504 residues long. In another embodiment, the above sequence is used as the source of the LLO fragment incorporated in a vaccine of the present invention. In another embodiment, an LLO AA sequence of methods and compositions of the present invention is a homologue of SEQ ID No: 1. In another embodiment, the LLO AA sequence is a variant of SEQ ID No: 1. In another embodiment, the LLO AA sequence is a fragment of SEQ ID No: 1. In another embodiment, the LLO AA sequence is an isoform of SEQ ID No: 1. Each possibility represents a separate embodiment of the present invention.

In one embodiment, an isoform is a peptide or protein that has the same function and similar (or identical) sequence to another peptide or protein, but is the product of a different gene. In one embodiment, a variant is something that differs from another in a minor way.

In another embodiment, an LLO protein fragment is utilized in compositions and methods of the present invention. In another embodiment, the N-terminal LLO fragment is an N-terminal fragment. In another embodiment, the N-terminal LLO fragment has the sequence:

MKKIMLVFITLILVSLPIAQQTEAKDASAFNKENSISSVAPPASPPASPKTPIEKKHADEIDKYIQGLD YNKNNVLVYHGDAVTNVPPRKGYKDGNEYIVVEKKKKSINQNNADIQVVNAISSLTYPGALVKA NSELVENQPDVLPVKRDSLTLSIDLPGMTNQDNKIVVKNATKSNVNNAVNTLVERNEKYAQAY SNVSAKIDYDDEMAYSESQLIAKFGTAFKAVNNSLNVNFGAISEGKMQEEVISFKQIYYNVNVNEP TRPSRFFGKAVTKEQLQALGVNAENPPAYISSVAYGRQVYLKLSTNSHSTKVKAAFDAAVSGKSV SGDVELTNIIKNSSFKAVIYGGSAKDEVQIIDGNLGDLRDILKKGATFNRETPGVPIATTNFLKDNE LAVIKNNSEYIETTSKAYTDGKINIDHSGGYVAQFNISWDEVNYD (SEQ ID NO: 2). In another embodiment, an LLO AA sequence of methods and compositions of the present invention comprises the sequence set forth in SEQ ID No: 2. In another embodiment, an LLO AA sequence is a homologue of SEQ ID No: 2. In another embodiment, the LLO AA sequence is a variant of SEQ ID No: 2. In another embodiment, the LLO AA sequence is a fragment of SEQ ID No: 2. In another embodiment, the LLO AA sequence is an isoform of SEQ ID No: 2. Each possibility represents a separate embodiment of the present invention.

In another embodiment, the LLO fragment has the sequence: MKKIMLVFITLUVSLPIAQQTEAKDASAFNKENSISSVAPPASPPASPKTPIEKKHADEIDKYIQGLD YNKNNVLVYHGDAVTNVPPRKGYKDGNEYIVVEKKKKSINQNNADIQVVNAISSLTYPGALVKA NSELVENQPDVLPVKRDSLTLSIDLPGMTNQDNKIVVKNATKSNVNNAVNTLVERNEKYAQAY SNVSAKIDYDDEMAYSESQLIAKFGTAFKAVNNSLNVNFGAISEGKMQEEVISFKQIYYNVNVNEP TRPSRFFGKAVTKEQLQALGVNAENPPAYISSVAYGRQVYLKLSTNSHSTKVKAAFDAAVSGKSV SGDVELTNIIKNSSFKAVIYGGSAKDEVQIIDGNLGDLRDLKKGATFNRETPGVPIAYTTNFLKD NE LAVIKNNSEYIETTSKAYTD (SEQ ID NO: 3). In another embodiment, an LLO AA sequence of methods and compositions of the present invention comprises the sequence set forth in SEQ ID No: 3. In another embodiment, an LLO AA sequence is a homologue of SEQ ID No: 3. In another embodiment, the LLO AA sequence is a variant of SEQ ID No: 3. In another embodiment, the LLO AA sequence is a fragment of SEQ ID No: 3. In another embodiment, the LLO AA sequence is an isoform of SEQ ID No: 3. Each possibility represents a separate embodiment of the present invention.

In another embodiment, the LLO fragment of methods and compositions of the present invention comprises a PEST-like domain. In another embodiment, an LLO fragment that comprises a PEST sequence is utilized.

In another embodiment, the LLO fragment does not contain the activation domain at the carboxy terminus. In another embodiment, the LLO fragment does not include cysteine 484. In another embodiment, the LLO fragment is a non-hemolytic fragment. In another embodiment, the LLO fragment is rendered non-hemolytic by deletion or mutation of the activation domain. In another embodiment, the LLO fragment is rendered non-hemolytic by deletion or mutation of cysteine 484. In another embodiment, the LLO fragment is rendered non-hemolytic by deletion or mutation at another location.

In another embodiment, the LLO fragment consists of about the first 441 AA of the LLO protein. In another embodiment, the LLO fragment comprises about the first 400-441 AA of the 529 AA full length LLO protein. In another embodiment, the LLO fragment corresponds to AA 1-441 of an LLO protein disclosed herein. In another embodiment, the LLO fragment consists of about the first 420 AA of LLO. In another embodiment, the LLO fragment corresponds to AA 1-420 of an LLO protein disclosed herein. In another embodiment, the LLO fragment consists of about AA 20-442 of LLO. In another embodiment, the LLO fragment corresponds to AA 20-442 of an LLO protein disclosed herein. In another embodiment, any ΔLLO without the activation domain comprising cysteine 484, and in particular without cysteine 484, are suitable for methods and compositions of the present invention.

In another embodiment, the LLO fragment corresponds to the first 400 AA of an LLO protein. In another embodiment, the LLO fragment corresponds to the first 300 AA of an LLO protein. In another embodiment, the LLO fragment corresponds to the first 200 AA of an LLO protein. In another embodiment, the LLO fragment corresponds to the first 100 AA of an LLO protein. In another embodiment, the LLO fragment corresponds to the first 50 AA of an LLO protein, which in one embodiment, comprises one or more PEST-like sequences.

In another embodiment, the LLO fragment contains residues of a homologous LLO protein that correspond to one of the above AA ranges. The residue numbers need not, in another embodiment, correspond exactly with the residue numbers enumerated above; e.g. if the homologous LLO protein has an insertion or deletion, relative to an LLO protein utilized herein.

Each LLO protein and LLO fragment represents a separate embodiment of the present invention.

In another embodiment of methods and compositions of the present invention, a fragment of an ActA protein is fused to the IgE fragment. In another embodiment, the fragment of an ActA protein has the sequence:

MRAMMVVFITANCITINPDIIFAATDSEDSSLNTDEWEEEKTEEQPSEVNTGPRYETAREVS SRDIKELEKSNKVRNTNKADLIAMLKEKAEKGPNINNNNSEQTENAAINEEASGADRPAIQVERRH PGLPSDSAAEIKKRRKAIASSDSELESLTYPDKPTKVNKKKVAKESVADASESDLDSSMQSADESS PQPLKANQQPFFPKVFKKIKDAGKWVRDKIDENPEVKKAIVDKSAGLIDQLLTKKKSEEVNASD FPPPPTDEELRLALPETPMLLGFNAPATSEPSSFEFPPPPTDEELRLALPETPMLLGFNAPATSEPS SFEFPPPPTEDELEIIRETASSLDSSFTRGDLASLRNAINRHSQNFSDFPPIPTEEELNGRGGRP (SEQ ID No: 4). In another embodiment, an ActA AA sequence of methods and compositions of the present invention comprises the sequence set forth in SEQ ID No: 4. In another embodiment, an ActA AA sequence is a homologue of SEQ ID No: 4. In another embodiment, the ActA AA sequence is a variant of SEQ ID No: 4. In another embodiment, the ActA AA sequence is a fragment of SEQ ID No: 4. In another embodiment, the ActA AA sequence is an isoform of SEQ ID No: 4. Each possibility represents a separate embodiment of the present invention.

In another embodiment, the ActA fragment is encoded by a recombinant nucleotide comprising the sequence:

ATGCGTGCGATGATGGTGGTTTTCATTACTGCCAATTGCATTACGATTAACCCCGACATAA TATTTGCAGCGACAGATAGCGAAGATTCTAGTCTAAACACAGATGAATGGGAAGAAGAAA AAACAGAAGAGCAACCAAGCGAGGTAAATACGGGACCAAGATACGAAACTGCACGTGAA GTAAGTTCACGTGATATTAAAGAACTAGAAAAATCGAATAAAGTGAGAAATACGAACAAA GCAGACCTAATAGCAATGTTGAAAGAAAAAGCAGAAAAAGGTCCAAATATCAATAATAAC AACAGTGAACAAACTGAGAATGCGGCTATAAATGAAGAGGCTTCAGGAGCCGACCGACCA GCTATACAAGTGGAGCGTCGTCATCCAGGATTGCCATCGGATAGCGCAGCGGAAATTAAAA AAAGAAGGAAAGCCATAGCATCATCGGATAGTGAGCTTGAAAGCCTTACTTATCCGGATAA ACCAACAAAAGTAAATAAGAAAAAAGTGGCGAAAGAGTCAGTTGCGGATGCTTCTGAAA GTGACTTAGATTCTAGCATGCAGTCAGCAGATGAGTCTTCACCACAACCTTTAAAAGCAAA CCAACAACCATTTTTCCCTAAAGTATTTAAAAAAATAAAAGATGCGGGGAAATGGGTACG TGATAAAATCGACGAAAATCCTGAAGTAAAGAAAGCGATTGTTGATAAAAGTGCAGGGTT AATTGACCAATTATTAACCAAAAAGAAAAGTGAAGAGGTAAATGCTTCGGACTTCCCGCC ACCACCTACGGATGAAGAGTTAAGACTTGCTTTGCCAGAGACACCAATGCTTCTTGGTTT AATGCTCCTGCTACATCAGAACCGAGCTCATTCGAATTTCCACCACCACCTACGGATGAAG AGTTAAGACTTGCTTTGCCAGAGACGCCAATGCTTCTTGGTTTTAATGCTCCTGCTACATCG GAACCGAGCTCGTTCGAATTTCCACCGCCTCCAACAGAAGATGAACTAGAAATCATCCGG GAAACAGCATCCTCGCTAGATTCTAGTTTTACAAGAGGGGATTTAGCTAGTTCGAGAAATG CTATTAATCGCCATAGTCAAAATTTCTCTGATTTCCCACCAATCCCAACAGAAGAAGAGTT GAACGGGAGAGGCGGTAGACCA (SEQ ID NO: 5). In another embodiment, the recombinant nucleotide has the sequence set forth in SEQ ID NO: 5. In another embodiment, an ActA-encoding nucleotide of methods and compositions of the present invention comprises the sequence set forth in SEQ ID No: 5. In another embodiment, the ActA-encoding nucleotide is a homologue of SEQ ID No: 5. In another embodiment, the ActA-encoding nucleotide is a variant of SEQ ID No: 5. In another embodiment, the ActA-encoding nucleotide is a fragment of SEQ ID No: 5. In another embodiment, the ActA-encoding nucleotide is an isoform of SEQ ID No: 5. Each possibility represents a separate embodiment of the present invention.

In another embodiment, the ActA fragment is any other ActA fragment known in the art. In another embodiment, a recombinant nucleotide of the present invention comprises any other sequence that encodes a fragment of an ActA protein. In another embodiment, the recombinant nucleotide comprises any other sequence that encodes an entire ActA protein. Each possibility represents a separate embodiment of the present invention.

In another embodiment of methods and compositions of the present invention, a PEST-like AA sequence is fused to the IgE fragment. In another embodiment, the PEST-like AA sequence is KENSISSMAPPASPPASPKTPIEKKHADEIDK (SEQ ID NO: 6). In another embodiment, the PEST-like sequence is KENSISSMAPPASPPASPK (SEQ ID No: 7). In another embodiment, fusion of an antigen to any LLO sequence, which in one embodiment, is one of the PEST-like AA sequences enumerated herein, can enhance cell mediated immunity against IgE.

In another embodiment, the PEST-like AA sequence is a PEST-like sequence from a Listeria ActA protein. In another embodiment, the PEST-like sequence is KTEEQPSEVNTGPR (SEQ ID NO: 8), KASVTDTSEGDLDSSMQSADESTPQPLK (SEQ ID NO: 9), KNEEVNASDFPPPPTDEELR (SEQ ID NO: 10), or RGGIPTSEEFSSLNSGDFTDDENSETTEEEIDR (SEQ ID NO: 11). In another embodiment, the PEST-like sequence is from Listeria seeligeri cytolysin, encoded by the Iso gene. In another embodiment, the PEST-like sequence is RSEVTISPAETPESPPATP (SEQ ID NO: 12). In another embodiment, the PEST-like sequence is from Streptolysin 0 protein of Streptococcus sp. In another embodiment, the PEST-like sequence is from Streptococcus pyogenes Streptolysin O, e.g. KQNTASTETTTTNEQPK (SEQ ID NO: 13) at AA 35-51. In another embodiment, the PEST-like sequence is from Streptococcus equisimilis Streptolysin O, e.g. KQNTANTETTTNEQPK (SEQ ID NO: 14) at AA 38-54. In another embodiment, the PEST-like sequence has a sequence selected from SEQ ID NO: 8-14. In another embodiment, the PEST-like sequence has a sequence selected from SEQ ID NO: 6-14. In another embodiment, the PEST-like sequence is another PEST-like AA sequence derived from a prokaryotic organism.

“PEST-like sequence” refers, in another embodiment, to a region rich in proline (P), glutamic acid (E), serine (S), and threonine (T) residues. In another embodiment, a PEST-like sequence is defined as a hydrophilic stretch of at least 12 AA in length with a high local concentration of proline (P), aspartate (D), glutamate (E), serine (S), and/or threonine (T) residues. In another embodiment, a PEST-like sequence contains no positively charged AA, namely arginine (R), histidine (H) and lysine (K). In another embodiment, the PEST-like sequence is flanked by one or more clusters containing several positively charged amino acids. In another embodiment, the PEST-like sequence mediates rapid intracellular degradation of proteins containing it. In another embodiment, the PEST-like sequence contains one or more internal phosphorylation sites, and phosphorylation at these sites precedes protein degradation.

In one embodiment, PEST-like sequences of prokaryotic organisms are identified in accordance with methods such as described by, for example Rechsteiner and Rogers (1996, Trends Biochem. Sci. 21:267-271) for LM and in Rogers S et al (Science 1986; 234(4774):364-8). Alternatively, PEST-like AA sequences from other prokaryotic organisms can also be identified based on this method. Other prokaryotic organisms wherein PEST-like AA sequences would be expected to include, but are not limited to, other Listeria species. In one embodiment, the PEST-like sequence fits an algorithm disclosed in Rogers et al. In another embodiment, the PEST-like sequence fits an algorithm disclosed in Rechsteiner et al. In another embodiment, the PEST-like sequence is identified using the PEST-find program.

In another embodiment, identification of PEST motifs is achieved by an initial scan for positively charged AA R, H, and K within the specified protein sequence. All AA between the positively charged flanks are counted and only those motifs are considered further, which contain a number of AA equal to or higher than the window-size parameter. In another embodiment, a PEST-like sequence must contain at least 1 P, 1 D or E, and at least 1 S or T.

In another embodiment, the quality of a PEST motif is refined by means of a scoring parameter based on the local enrichment of critical AA as well as the motif's hydrophobicity. Enrichment of D, E, P, S and T is expressed in mass percent (w/w) and corrected for 1 equivalent of D or E, 1 of P and 1 of S or T. In another embodiment, calculation of hydrophobicity follows in principle the method of J. Kyte and R. F. Doolittle (Kyte, J and Dootlittle, R F. J. Mol. Biol. 157, 105 (1982). For simplified calculations, Kyte-Doolittle hydropathy indices, which originally ranged from −4.5 for arginine to +4.5 for isoleucine, are converted to positive integers, using the following linear transformation, which yielded values from 0 for arginine to 90 for isoleucine.

Hydropathy index=10*Kyte-Doolittle hydropathy index+45

In another embodiment, a potential PEST motif's hydrophobicity is calculated as the sum over the products of mole percent and hydrophobicity index for each AA species. The desired PEST score is obtained as combination of local enrichment term and hydrophobicity term as expressed by the following equation:

PESTscore=0.55*DEPST−0.5*hydrophobicity index.

In another embodiment, “PEST-like sequence” or “PEST-like sequence peptide” refers to a peptide having a score of at least +5, using the above algorithm. In another embodiment, the term refers to a peptide having a score of at least 6. In another embodiment, the peptide has a score of at least 7. In another embodiment, the score is at least 8. In another embodiment, the score is at least 9. In another embodiment, the score is at least 10. In another embodiment, the score is at least 11. In another embodiment, the score is at least 12. In another embodiment, the score is at least 13. In another embodiment, the score is at least 14. In another embodiment, the score is at least 15. In another embodiment, the score is at least 16. In another embodiment, the score is at least 17. In another embodiment, the score is at least 18. In another embodiment, the score is at least 19. In another embodiment, the score is at least 20. In another embodiment, the score is at least 21. In another embodiment, the score is at least 22. In another embodiment, the score is at least 22. In another embodiment, the score is at least 24. In another embodiment, the score is at least 24. In another embodiment, the score is at least 25. In another embodiment, the score is at least 26. In another embodiment, the score is at least 27. In another embodiment, the score is at least 28. In another embodiment, the score is at least 29. In another embodiment, the score is at least 30. In another embodiment, the score is at least 32. In another embodiment, the score is at least 35. In another embodiment, the score is at least 38. In another embodiment, the score is at least 40. In another embodiment, the score is at least 45. Each possibility represents a separate embodiment of the present invention.

In another embodiment, the PEST-like sequence is identified using any other method or algorithm known in the art, e.g the CaSPredictor (Garay-Malpartida H M, Occhiucci J M, Alves J, Belizario J E. Bioinformatics. 2005 Jun., 21 Suppli 1:1169-76). In another embodiment, the following method is used:

A PEST index is calculated for each stretch of appropriate length (e.g. a 30-35 AA stretch) by assigning a value of 1 to the AA Ser, Thr, Pro, Glu, Asp, Asn, or Gln. The coefficient value (CV) for each of the PEST residue is 1 and for each of the other AA (non-PEST) is 0.

Each method for identifying a PEST-like sequence represents a separate embodiment of the present invention.

In another embodiment, the PEST-like sequence is any other PEST-like sequence known in the art. Each PEST-like sequence and type thereof represents a separate embodiment of the present invention.

“Fusion to a PEST-like sequence” refers, in another embodiment, to fusion to a protein fragment comprising a PEST-like sequence. In another embodiment, the term includes cases wherein the protein fragment comprises surrounding sequence other than the PEST-like sequence. In another embodiment, the protein fragment consists of the PEST-like sequence. Thus, in another embodiment, “fusion” refers to two peptides or protein fragments either linked together at their respective ends or embedded one within the other. Each possibility represents a separate embodiment of the present invention.

In another embodiment, fusion proteins of the present invention are prepared by a process comprising subcloning of appropriate sequences, followed by expression of the resulting nucleotide. In another embodiment, subsequences are cloned and the appropriate subsequences cleaved using appropriate restriction enzymes. The fragments are then ligated, in another embodiment, to produce the desired DNA sequence. In another embodiment, DNA encoding the fusion protein is produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The insert is then ligated into a plasmid. In another embodiment, a similar strategy is used to produce a protein wherein an IgE protein fragment is embedded within a heterologous peptide.

In one embodiment, ActA, LLO and/or PEST-like sequences fused to a peptide such as HPV E7 increased the immune response to said peptide (Example 2), conferred antitumor immunity (Examples 1 and 3), and generated peptide-specific CD8+ cells (Examples 2 and 3), even if the fusion peptide was expressed in a non-Listeria vector (Example 4). In one embodiment, LLO and/or PEST-like sequences fused to a peptide which is a self-antigen, which in one embodiment, is an antigen that it is endogenously produced by the organism, increased the immune response to said self-antigen (Examples 5-7).

In another embodiment, a recombinant polypeptide of the present invention is made by a process comprising the step of chemically conjugating a first polypeptide comprising an IgE fragment to a second polypeptide comprising a non-IgE AA sequence. In another embodiment, an IgE fragment is conjugated to a second polypeptide comprising the non-IgE AA sequence. In another embodiment, a peptide comprising an IgE fragment is conjugated to a non-IgE AA sequence. In another embodiment, an IgE fragment is conjugated to a non-IgE AA sequence. Each possibility represents a separate embodiment of the present invention.

The IgE fragment of methods and compositions of the present invention is, in another embodiment, a C epsilon-1 domain. In another embodiment, the IgE fragment is a C epsilon-2 domain. In another embodiment, the IgE fragment is a C epsilon-3 domain. In another embodiment, the IgE fragment is a C epsilon-4 domain. In another embodiment, the IgE fragment is an M1 domain. In another embodiment, the IgE fragment is a M2 domain. In another embodiment, the IgE fragment is an M1/M2 domain. In another embodiment, the IgE fragment includes more than 1 of the above domains (e.g. C epsilon-1 and C epsilon-2). In another embodiment, the IgE fragment is a fragment of 1 of the above domains. In another embodiment, the IgE fragment overlaps with, but does not entirely include, 1 of the above domains (e.g. the region contains part of the C epsilon-3 domain). In another embodiment, the IgE fragment overlaps with more than 1 of the above domains (e.g. part of the M1 domain and part of the M2 domain). In another embodiment, the IgE fragment is any other region or fragment of IgE known in the art. Each possibility represents a separate embodiment of the present invention.

“M1 domain,” “M2 domain,” and “M1/M2 domain” refer, in another embodiment, to domains encoded by the M1, M2, and M l+M2 exons, respectively. In another embodiment, the terms refer to IgE fragments that overlap with one of the above domains.

In another embodiment, the IgE protein of methods and compositions of the present invention is a human IgE protein. In another embodiment, the protein is a mouse IgE protein. In another embodiment, the protein is derived from any other species know in the art. Each possibility represents a separate embodiment of the present invention.

In another embodiment, an IgE fragment of methods and compositions of the present invention is fragment of the sequence:

MDWTWILFLVAAATRVHSQTQLVQSGAEVRKPGASVRVSCKASGYTFIDSYIHWIRQAPG HGLEWVGWINPNSGGTNYAPRFQGRVTMTRDASFSTAYMDLRSLRSDDSAVFYCAKSDPFW SDYYNFDYSYTLDVWGQGTTVTVSSASTQSPSVFPLTRCCKNIPSNATSVTLGCLATGYFPEPV MVTWDTGSLNGTTMTLPATTLTLSGHYATISLLTVSGAWAKQMFTCRVAHTPSSTDWVDNKT FSVCSRDFTPPTVKILQSSCDGGGHFPPTIQLLCLVSGYTPGTINITWLEDGQVMDVDLSTASTT QEGELASTQSELTLSQKHWLSDRTYTCQVTYQGHTFEDSTKKCADSNPRGVSAYLSRPSPFDL FIRKSPTITCLVVDLAPSKGTVNLTWSRASGKPVNHSTRKEEKQRNGTLTVTSTLPVGTRDWIE GETYQCRVTHPHLPRALMRSTTKTSGPRAAPEVYAFATPEWPGSRDKRTLACLIQNFMPEDIS VQWLHNEVQLPDARHSTTQPRKTKGSGFFVFSRLEVTRAEWEQKDEFICRAVHEAASPSQTV QRAVSVNPGK (SEQ ID No: 15; GenBank Accession Number L00022). In another embodiment, the IgE fragment is a fragment of SEQ ID No: 15. In another embodiment, the IgE fragment is a fragment of a homologue of SEQ ID No: 15. In another embodiment, the IgE fragment is a fragment of a variant of SEQ ID No: 15. In another embodiment, the IgE fragment is a fragment of an isoform of SEQ ID No: 15. Each possibility represents a separate embodiment of the present invention.

In another embodiment, an IgE fragment of methods and compositions of the present invention is a fragment of the AA sequence encoded by the nucleotide sequence set forth in SEQ ID No: 16 (Example 9). In another embodiment, the IgE fragment is encoded by a fragment of a human homologue of SEQ ID No: 16. In another embodiment, the IgE fragment is encoded by a fragment of a variant of SEQ ID No: 16. In another embodiment, the IgE fragment is encoded by a fragment of an isoform of SEQ ID No: 16. In another embodiment, the IgE fragment is encoded by a fragment of a variant of a human homologue of SEQ ID No: 16. In another embodiment, the IgE fragment is encoded by a fragment of an isoform of a human homologue of SEQ ID No: 16. Each possibility represents a separate embodiment of the present invention.

In another embodiment, an IgE fragment of methods and compositions of the present invention has the AA sequence:

TVTWYSDSLNMSTVNFPALGSELKVTTSQVTSWGKSAKNFfCHVTHPPSFNESRTILVRPV NITEPTLELLHSSCDPNAFHSTIQLYCFIYGHILNDVSVSWLMDDREITDTLAQTVLIKEEGKLAS TCSKLNITEQQWMSESTFTCKVTSQGVDYLAHTRRCPDHEPRGVITYLPPSPLDLYQNGAPKLT CLVVDLESEKNVNVTWNQEKKTSVSASQWYTKHHNNATTSITSILPVVAKDWIEGYGYQCIVD HPDFPKPIVRSIKTPGQRSAPEVYVFPPPEEESEDKRTLTCLIQNFFPEDISVQWLGDGKLISNSQ HSTTTPLKSNGSNQGFFIFSRLEVAKTLWTQRKQFTCQVIHEALQKPRKLEKTISTSLGNTSLRPS (SEQ ID No: 17). In another embodiment, the IgE fragment is a fragment of SEQ ID No: 17. In another embodiment, the IgE fragment is a fragment of a homologue of SEQ ID No: 17. In another embodiment, the IgE fragment is a fragment of a variant of SEQ ID No: 17. In another embodiment, the IgE fragment is a fragment of an isoform of SEQ ID No: 17. Each possibility represents a separate embodiment of the present invention.

In another embodiment, the IgE fragment of methods and compositions of the present invention is encoded by a nucleotide molecule having the sequence:

aggctgatttttgaagaaaggggttgtagcctaaaagatgatggtgttaagtcttctgtacctgttgacagcccttccgggtatcctgtcaga ggtgcagcttcaggagtcaggacctagcctcgtgaaaccttctcagactctgtccctcacatgttctgtcactggcgactccatcaccagtggttactgg aactggatccggcaagtcccagggaataaacttgagtacatgggtttcataaattacagtggtaacacttactacaatccatctctgagaagtcgaatct ccatcactcgagacacatccaagaaccagtacttcctgcacttgaattctgtgactactgaggacacagccacatattactgtgcaagggctaactggg acgtctttgcttactggggcaagggactctggtcactgtctctgca (sequence encoding heavy chain from IgELa2; SEQ ID No: 18). In another embodiment, the IgE fragment is a fragment of SEQ ID No: 18. In another embodiment, the IgE fragment is encoded by a fragment of a human homologue of SEQ ID No: 18. In another embodiment, the IgE fragment is encoded by a fragment of a variant of SEQ ID No: 18. In another embodiment, the IgE fragment is encoded by a fragment of an isoform of SEQ ID No: 18. In another embodiment, the IgE fragment is encoded by a fragment of a variant of a human homologue of SEQ ID No: 18. In another embodiment, the IgE fragment is encoded by a fragment of an isoform of a human homologue of SEQ ID No: 18. Each possibility represents a separate embodiment of the present invention.

In another embodiment, the IgE fragment of methods and compositions of the present invention has the AA sequence:

MMVLSLLYLLTALPGILSEVQLQESGPSLVKPSQTLSLTCSVTGDSITSGYWNWIRQVPGNK LEYMGFINYSGNTYYNPSLRSRISLRDTSKNQYFLHLNSVTTEDTATYYCARANWDVFAYWGQG TLVTVSA (heavy chain from IgELa2; SEQ ID No: 19). In another embodiment, the IgE fragment is a fragment of SEQ ID No: 19. In another embodiment, the IgE fragment is a fragment of a homologue of SEQ ID No: 19. In another embodiment, the IgE fragment is a fragment of a variant of SEQ ID No: 19. In another embodiment, the IgE fragment is a fragment of an isoform of SEQ ID No: 19. Each possibility represents a separate embodiment of the present invention.

In another embodiment, a cDNA of an alternatively spliced IgE isoform is administered in a vaccine of the present invention. In another embodiment, a fragment of a cDNA of an alternatively spliced IgE isoform is administered. Alternatively spliced IgE isoform are well known in the art, and are described, for example, in Batista F D et al (Characterization of a second secreted IgE isoform and identification of an asymmetric pathway of IgE assembly. Proc Natl Acad Sci USA. 1996 Apr. 16; 93(8):3399-404) and Lyczak J B et al (Expression of novel secreted isoforms of human immunoglobulin E proteins. J Biol. Chem. 1996 Feb. 16; 271(7):3428-36). Each isoform and each fragment thereof represents a separate embodiment of the present invention.

In another embodiment, the IgE fragment is any other fragment of any other IgE protein known in the art.

In another embodiment, the IgE fragment of methods and compositions of the present invention is fused to the non-IgE AA sequence. In another embodiment, the IgE fragment is embedded within the non-IgE AA sequence. In another embodiment, an IgE-derived peptide is incorporated into an LLO fragment, ActA protein or fragment, or PEST-like sequence, as exemplified herein (DP-L2851, Example 8). Each possibility represents a separate embodiment of the present invention.

In another embodiment, an IgE fragment of methods and compositions of the present invention is smaller than about 400 residues. In another embodiment, an IgE fragment of methods and compositions of the present invention is smaller than about 14 kDa. In another embodiment, an IgE fragment of methods and compositions of the present invention is smaller than about 60 kD, while in another embodiment, it is smaller than about 50 kD, while in another embodiment, it is smaller than about 25 kD. In another embodiment, an IgE fragment of methods and compositions of the present invention is a size that allows it to be readily secreted by a recombinant Listeria strain.

In another embodiment, the length of the IgE fragment of the present invention is at least 8 amino acids (AA). In another embodiment, the length is more than 8 AA. In another embodiment, the length is at least 9 AA. In another embodiment, the length is more than 9 AA. In another embodiment, the length is at least 10 AA. In another embodiment, the length is more than 10 AA. In another embodiment, the length is at least 11 AA. In another embodiment, the length is more than 11 AA. In another embodiment, the length is at least 12 AA. In another embodiment, the length is more than 12 AA. In another embodiment, the length is at least about 14 AA. In another embodiment, the length is more than 14 AA. In another embodiment, the length is at least about 16 AA. In another embodiment, the length is more than 16 AA. In another embodiment, the length is at least about 18 AA. In another embodiment, the length is more than 18 AA. In another embodiment, the length is at least about 20 AA. In another embodiment, the length is more than 20 AA. In another embodiment, the length is at least about 25 AA. In another embodiment, the length is more than 25 AA. In another embodiment, the length is at least about 30 AA. In another embodiment, the length is more than 30 AA. In another embodiment, the length is at least about 40 AA. In another embodiment, the length is more than 40 AA. In another embodiment, the length is at least about 50 AA. In another embodiment, the length is more than 50 AA. In another embodiment, the length is at least about 70 AA. In another embodiment, the length is more than 70 AA. In another embodiment, the length is at least about 100 AA. In another embodiment, the length is more than 100 AA. In another embodiment, the length is at least about 150 AA. In another embodiment, the length is more than 150 AA. In another embodiment, the length is at least about 200 AA. In another embodiment, the length is more than 200 AA. Each possibility represents a separate embodiment of the present invention.

In another embodiment, the length is about 8-50 AA. In another embodiment, the length is about 8-70 AA. In another embodiment, the length is about 8-100 AA. In another embodiment, the length is about 8-150 AA. In another embodiment, the length is about 8-200 AA. In another embodiment, the length is about 8-250 AA. In another embodiment, the length is about 8-300 AA. In another embodiment, the length is about 8-400 AA. In another embodiment, the length is about 8-500 AA. In another embodiment, the length is about 9-50 AA. In another embodiment, the length is about 9-70 AA. In another embodiment, the length is about 9-100 AA. In another embodiment, the length is about 9-150 AA. In another embodiment, the length is about 9-200 AA. In another embodiment, the length is about 9-250 AA. In another embodiment, the length is about 9-300 AA. In another embodiment, the length is about 10-50 AA. In another embodiment, the length is about 10-70 AA. In another embodiment, the length is about 10-100 AA. In another embodiment, the length is about 10-150 AA. In another embodiment, the length is about 10-200 AA. In another embodiment, the length is about 10-250 AA. In another embodiment, the length is about 10-300 AA. In another embodiment, the length is about 10-400 AA. In another embodiment, the length is about 10-500 AA. In another embodiment, the length is about 11-50 AA. In another embodiment, the length is about 11-70 AA. In another embodiment, the length is about 11-100 AA. In another embodiment, the length is about 11-150 AA. In another embodiment, the length is about 11-200 AA: In another embodiment, the length is about 11-250 AA. In another embodiment, the length is about 11-300 AA. In another embodiment, the length is about 11-400 AA. In another embodiment, the length is about 1′-500 AA. In another embodiment, the length is about 12-50 AA. In another embodiment, the length is about 12-70 AA. In another embodiment, the length is about 12-100 AA. In another embodiment, the length is about 12-150 AA. In another embodiment, the length is about 12-200 AA. In another embodiment, the length is about 12-250 AA. In another embodiment, the length is about 12-300 AA. In another embodiment, the length is about 12-400 AA. In another embodiment, the length is about 12-500 AA. In another embodiment, the length is about 15-50 AA. In another embodiment, the length is about 15-70 AA. In another embodiment, the length is about 15-100 AA. In another embodiment, the length is about 15-150 AA. In another embodiment, the length is about 15-200 AA. In another embodiment, the length is about 15-250 AA. In another embodiment, the length is about 15-300 AA. In another embodiment, the length is about 15-400 AA. In another embodiment, the length is about 15-500 AA. In another embodiment, the length is about 8-400 AA. In another embodiment, the length is about 8-500 AA. In another embodiment, the length is about 20-50 AA. In another embodiment, the length is about 20-70 AA. In another embodiment, the length is about 20-100 AA. In another embodiment, the length is about 20-150 AA. In another embodiment, the length is about 20-200 AA. In another embodiment, the length is about 20-250 AA. In another embodiment, the length is about 20-300 AA. In another embodiment, the length is about 20-400 AA. In another embodiment, the length is about 20-500 AA. In another embodiment, the length is about 30-50 AA. In another embodiment, the length is about 30-70 AA. In another embodiment, the length is about 30-100 AA. In another embodiment, the length is about 30-150 AA. In another embodiment, the length is about 30-200 AA. In another embodiment, the length is about 30-250 AA. In another embodiment, the length is about 30-300 AA. In another embodiment, the length is about 30-400 AA. In another embodiment, the length is about 30-500 AA. In another embodiment, the length is about 40-50 AA. In another embodiment, the length is about 40-70 AA. In another embodiment, the length is about 40-100 AA. In another embodiment, the length is about 40-150 AA. In another embodiment, the length is about 40-200 AA. In another embodiment, the length is about 40-250 AA. In another embodiment, the length is about 40-300 AA. In another embodiment, the length is about 40-400 AA. In another embodiment, the length is about 40-500 AA. In another embodiment, the length is about 50-70 AA. In another embodiment, the length is about 50-100 AA. In another embodiment, the length is about 50-150 AA. In another embodiment, the length is about 50-200 AA. In another embodiment, the length is about 50-250 AA. In another embodiment, the length is about 50-300 AA. In another embodiment, the length is about 50-400 AA. In another embodiment, the length is about 50-500 AA. In another embodiment, the length is about 70-100 AA. In another embodiment, the length is about 70-150 AA. In another embodiment, the length is about 70-200 AA. In another embodiment, the length is about 70-250 AA. In another embodiment, the length is about 70-300 AA. In another embodiment, the length is about 70-400 AA. In another embodiment, the length is about 70-500 AA. In another embodiment, the length is about 100-150 AA. In another embodiment, the length is about 100-200 AA. In another embodiment, the length is about 100-250 AA. In another embodiment, the length is about 100-300 AA. In another embodiment, the length is about 100-400 AA. In another embodiment, the length is about 100-500 AA. Each possibility represents a separate embodiment of the present invention.

In another embodiment, a recombinant polypeptide of methods and compositions of the present invention comprises a signal sequence. In another embodiment, the signal sequence is from the organism used to construct the vaccine vector. In another embodiment, the signal sequence is a LLO signal sequence. In another embodiment, the signal sequence is an ActA signal sequence. In another embodiment, the signal sequence is a Listerial signal sequence. In another embodiment, the signal sequence is any other signal sequence known in the art. Each possibility represents a separate embodiment of the present invention.

The terms “peptide” and “recombinant peptide” refer, in another embodiment, to a peptide or polypeptide of any length. In another embodiment, a peptide or recombinant peptide of the present invention has one of the lengths enumerated above for an IgE fragment. Each possibility represents a separate embodiment of the present invention.

In one embodiment, the term “peptide” refers to native peptides (either degradation products, synthetically synthesized peptides or recombinant peptides) and/or peptidomimetics (typically, synthetically synthesized peptides), such as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH₃)—CO—), ester bonds (—C(R)H—C—O—O—C(R)—N—), ketomethylen bonds (—CO—CH₂—), *-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom.

These modifications can occur at any of the bonds along the peptide chain and even at several (2-3) at the same time. Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

In one embodiment, the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodemosine, nor-valine, nor-leucine and ornithine. Furthermore, the term “amino acid” may include both D- and L-amino acids.

Peptides or proteins of this invention may be prepared by various techniques known in the art, including phage display libraries [Hoogenboom and Winter, J. Mol. Biol. 227:381 (1991); Marks et al., J. Mol. Biol. 222:581 (1991)].

In one embodiment, the term “oligonucleotide” is interchangeable with the term “nucleic acid”, and may refer to a molecule, which may include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also refers to sequences that include any of the known base analogs of DNA and RNA.

In another embodiment, the present invention provides a vaccine comprising a recombinant polypeptide of the present invention and an adjuvant.

In another embodiment, the present invention provides an immunogenic composition comprising a recombinant polypeptide of the present invention. In another embodiment, the immunogenic composition of methods and compositions of the present invention comprises a recombinant vaccine vector encoding a recombinant peptide of the present invention. In another embodiment, the immunogenic composition comprises a plasmid encoding a recombinant peptide of the present invention. In another embodiment, the immunogenic composition comprises an adjuvant. Each possibility represents a separate embodiment of the present invention.

An immunogenic composition of methods and compositions of the present invention comprises, in another embodiment, an adjuvant that favors a predominantly Th1-type immune response. In another embodiment, the adjuvant favors a predominantly Th1-mediated immune response. In another embodiment, the adjuvant favors a Th1-type immune response. In another embodiment, the adjuvant favors a Th1-mediated immune response. In another embodiment, the adjuvant favors a cell-mediated immune response over an antibody-mediated response. In another embodiment, the adjuvant is any other type of adjuvant known in the art. In another embodiment, the immunogenic composition induces the formation of a T cell immune response against the target IgE protein. Each possibility represents a separate embodiment of the present invention.

In another embodiment, the adjuvant is MPL. In another embodiment, the adjuvant is QS21. In another embodiment, the adjuvant is a TLR agonist. In another embodiment, the adjuvant is a TLR4 agonist. In another embodiment, the adjuvant is a TLR9 agonist. In another embodiment, the adjuvant is Resiquimod®. In another embodiment, the adjuvant is imiquimod. In another embodiment, the adjuvant is a CpG oligonucleotide. In another embodiment, the adjuvant is a cytokine or a nucleotide molecule encoding same. In another embodiment, the adjuvant is a chemokine or a nucleotide molecule encoding same. In another embodiment, the adjuvant is IL-12 or a nucleotide molecule encoding same. In another embodiment, the adjuvant is IL-6 or a nucleotide molecule encoding same. In another embodiment, the adjuvant is a lipopolysaccharide. In another embodiment, the adjuvant is any other adjuvant known in the art. Each possibility represents a separate embodiment of the present invention.

“Predominantly Th1-type immune response” refers, in another embodiment, to an immune response in which more than 60% of the antigen-specific CD4⁺ T cells detectable by a standard method are Th1-type T cells. In another embodiment, more than 70% of the detectable antigen-specific CD 4⁺T cells are Th1-type. In another embodiment, more than 80% of the detectable antigen-specific CD4⁺ T cells are Th1-type. In another embodiment, more than 85% of the detectable antigen-specific CD4⁺ T cells are Th1-type. In another embodiment, more than 90% of the detectable antigen-specific CD4⁺ T cells are Th1-type. In another embodiment, more than 95% of the detectable antigen-specific CD4⁺ T cells are Th1-type. In another embodiment, more than 97% of the detectable antigen-specific CD4⁺ T cells are Th1-type. In another embodiment, more than 99% of the detectable antigen-specific CD4⁺ T cells are Th1-type. In another embodiment, there are no detectable antigen-specific Th2-type CD4⁺ T cells. In another embodiment, only background levels of antigen-specific Th2-type CD4⁺ T cells are detected.

In another embodiment, a “predominantly Th1-type immune response” refers to an immune response in which IFN-gamma is secreted. In another embodiment, it refers to an immune response in which tumor necrosis factor-β is secreted. In another embodiment, it refers to an immune response in which IL-2 is secreted. Each possibility represents a separate embodiment of the present invention.

“Favors” a predominantly Th1-type immune response refers, in another embodiment, to induction of a predominantly Th1-type immune response in a majority of subjects tested. In another embodiment, the term refers to an induction of a predominantly Th1-type immune response in over 60% of subjects tested. In another embodiment, the number is over 70%. In another embodiment, the number is over 80%. In another embodiment, the number is over 85%. In another embodiment, the number is over 90%. In another embodiment, the number is over 95%. In another embodiment, the number is over 98%. In another embodiment, the number is 100%. In another embodiment, the number is 60%. In another embodiment, the number is 70%. In another embodiment, the number is 80%. In another embodiment, the number is 85%. In another embodiment, the number is 90%. In another embodiment, the number is 95%. In another embodiment, the number is 98%. Each possibility represents a separate embodiment of the present invention.

The method used to measure levels of Th1- and Th2-type T cells is, in another embodiment, fluorescence-activated cell sorting (FACS). In another embodiment, the method is any other method known in the art. Methods of measuring immune responses and levels of Th1 and Th2 T cells and cytotoxic T lymphocytes (CTL) are well known in the art, and include, for example, flow cytometry, target cell lysis assays (in another embodiment, chromium release assay) the use of tetramers, and others; these included methods for determining cell phenotype, genetic restriction, and fine specificity of recognition of responses. These methods are described, for example, in Current Protocols in Immunology (John E. Coligan et al, 02006 by John Wiley & Sons, Inc). In another embodiment, a method of measuring an immune response comprises in vitro antigen presentation to T cells and/or expansion of antigen-specific CTL. Methods for in vitro antigen presentation and/or CTL expansion are well known in the art, and are described, for example, in Sheil et al (Identification of an autologous insulin B chain peptide as a target antigen for H-2 Kb-restricted cytotoxic T lymphocytes. J Exp Med. 1992 Feb. 1; 175(2):545-52) and Carbone et al (Induction of cytotoxic T lymphocytes by primary in vitro stimulation with peptides. J Exp Med. 1988 Jun. 1; 167(6):1767-79). Each method represents a separate embodiment of the present invention.

The immunogenic composition utilized in methods and compositions of the present invention comprises, in another embodiment, a recombinant vaccine vector. In another embodiment, the recombinant vaccine vector comprises a recombinant peptide of the present invention. In another embodiment, the recombinant vaccine vector comprises a nucleotide molecule of the present invention. In another embodiment, the recombinant vaccine vector comprises a nucleotide molecule encoding a recombinant peptide of the present invention. Each possibility represents a separate embodiment of the present invention.

In another embodiment, the present invention provides a recombinant Listeria strain expressing a peptide, the peptide comprising a fragment of an IgE constant region.

In another embodiment, the present invention provides a recombinant vaccine vector encoding a recombinant polypeptide of the present invention. In another embodiment, the present invention provides a recombinant vaccine vector comprising a recombinant polypeptide of the present invention. In another embodiment, the expression vector is a plasmid. Methods for constructing and utilizing recombinant vectors are well known in the art and are described, for example, in Sambrook et al. (2001, Molecular Cloning: A Laboratory Manual, Cold Spring Harbor Laboratory, New York), and in Brent et al. (2003, Current Protocols in Molecular Biology, John Wiley & Sons, New York). Each possibility represents a separate embodiment of the present invention.

In another embodiment, the vector is an intracellular pathogen. In another embodiment, the vector is derived from a cytosolic pathogen. In another embodiment, the vector is derived from an intracellular pathogen. In another embodiment, an intracellular pathogen induces a predominantly cell-mediated immune response. In another embodiment, the vector is a Salmonella strain. In another embodiment, the vector is a BCG strain. In another embodiment, the vector is a bacterial vector. In another embodiment, the use of an intracellular pathogen does not induce antigen-specific Th2-type cells, thus reducing the possibility that that IgE-producing B cells will undergo polyclonal expansion (e.g. expansion induced by IL-4 secretion by Th2 CD4⁺ cells). In another embodiment, the recombinant vaccine vector does not induce a significant antibody response. In another embodiment, the recombinant vaccine vector induces a predominantly Th1-type immune response. Each possibility represents a separate embodiment of the present invention.

In another embodiment, the vector is selected from Salmonella sp., Shigella sp., BCG, L. monocytogenes, E. coli and S. gordonii. In another embodiment, the fusion proteins are delivered by recombinant bacterial vectors modified to escape phagolysosomal fusion and live in the cytoplasm of the cell. In another embodiment, the vector is a viral vector. In other embodiments, the vector is selected from Vaccinia, Avipox, Adenovirus, AAV, Vaccinia virus NYVAC, Modified vaccinia strain Ankara (MVA), Semliki Forest virus, Venezuelan equine encephalitis virus, herpes viruses, and retroviruses. In another embodiment, the vector is a naked DNA vector. In another embodiment, the vector is any other vector known in the art. Each possibility represents a separate embodiment of the present invention.

In another embodiment, the present invention provides a nucleotide molecule encoding a recombinant polypeptide of the present invention.

In another embodiment, the present invention provides a vaccine comprising a recombinant nucleotide molecule of the present invention and an adjuvant.

In another embodiment, the present invention provides a recombinant vaccine vector comprising a recombinant nucleotide molecule of the present invention.

In another embodiment, the present invention provides a recombinant Listeria strain comprising a recombinant nucleotide molecule of the present invention.

The recombinant Listeria strain of methods and compositions of the present invention is, in another embodiment, a recombinant Listeria monocytogenes strain. In another embodiment, the Listeria strain is a recombinant Listeria seeligeri strain. In another embodiment, the Listeria strain is a recombinant Listeria grayi strain. In another embodiment, the Listeria strain is a recombinant Listeria ivanovii strain. In another embodiment, the Listeria strain is a recombinant Listeria murrayi strain. In another embodiment, the Listeria strain is a recombinant Listeria welshimeri strain. In another embodiment, the Listeria strain is a recombinant strain of any other Listeria species known in the art.

In another embodiment the Listeria strain is attenuated by deletion of a gene. In another embodiment the Listeria strain is attenuated by deletion of more than 1 gene. In another embodiment the Listeria strain is attenuated by deletion or inactivation of a gene. In another embodiment the Listeria strain is attenuated by deletion or inactivation of more than 1 gene.

In another embodiment, the gene that is mutated is hly. In another embodiment, the gene that is mutated is actA. In another embodiment, the gene that is mutated is plc A. In another embodiment, the gene that is mutated is plcB. In another embodiment, the gene that is mutated is mpl. In another embodiment, the gene that is mutated is inl A. In another embodiment, the gene that is mutated is inlB. In another embodiment, the gene that is mutated is bsh.

In another embodiment, the Listeria strain is an auxotrophic mutant. In another embodiment, the Listeria strain is deficient in a gene encoding a vitamin synthesis gene. In another embodiment, the Listeria strain is deficient in a gene encoding pantothenic acid synthase.

In another embodiment, the Listeria strain is deficient in an AA metabolism enzyme. In another embodiment the Listeria strain is deficient in a D-glutamic acid synthase gene. In another embodiment the Listeria strain is deficient in the dat gene. In another embodiment the Listeria strain is deficient in the dal gene. In another embodiment the Listeria strain is deficient in the dga gene. In another embodiment the Listeria strain is deficient in a gene involved in the synthesis of diaminopimelic acid. CysK. In another embodiment, the gene is vitamin-B12 independent methionine synthase. In another embodiment, the gene is trpA. In another embodiment, the gene is trpB. In another embodiment, the gene is trpE. In another embodiment, the gene is asnB. In another embodiment, the gene is gltD. In another embodiment, the gene is gltB. In another embodiment, the gene is leuA. In another embodiment, the gene is argG. In another embodiment, the gene is thrC. In another embodiment, the Listeria strain is deficient in one or more of the genes described hereinabove.

In another embodiment, the Listeria strain is deficient in a synthase gene. In another embodiment, the gene is an AA synthesis gene. In another embodiment, the gene is folP. In another embodiment, the gene is dihydrouridine synthase family protein. In another embodiment, the gene is ispD. In another embodiment, the gene is ispF. In another embodiment, the gene is phosphoenolpyruvate synthase. In another embodiment, the gene is hisF. In another embodiment, the gene is his H. In another embodiment, the gene is fliI. In another embodiment, the gene is ribosomal large subunit pseudouridine synthase. In another embodiment, the gene is ispD. In another embodiment, the gene is bifunctional GMP synthase/glutamine amidotransferase protein. In another embodiment, the gene is cobS. In another embodiment, the gene is cobB. In another embodiment, the gene is cbiD. In another embodiment, the gene is uroporphyrin-III C-methyltransferase/uroporphyrinogen-III synthase. In another embodiment, the gene is cobQ. In another embodiment, the gene is uppS. In another embodiment, the gene is truB. In another embodiment, the gene is dxs. In another embodiment, the gene is mvaS. In another embodiment, the gene is dapA. In another embodiment, the gene is ispG. In another embodiment, the gene is folC. In another embodiment, the gene is citrate synthase. In another embodiment, the gene is argJ. In another embodiment, the gene is 3-deoxy-7-phosphoheptulonate synthase. In another embodiment, the gene is indole-3-glycerol-phosphate synthase. In another embodiment, the gene is anthranilate synthase/glutamine amidotransferase component. In another embodiment, the gene is menB. In another embodiment, the gene is menaquinone-specific isochorismate synthase. In another embodiment, the gene is phosphoribosylformylglycinamidine synthase I or II. In another embodiment, the gene is phosphoribosylaminoimidazole-succinocarboxamide synthase. In another embodiment, the gene is carB. In another embodiment, the gene is carA. In another embodiment, the gene is thyA. In another embodiment, the gene is mgsA. In another embodiment, the gene is aroB. In another embodiment, the gene is hepB. In another embodiment, the gene is rluB. In another embodiment, the gene is ilvB. In another embodiment, the gene is ilvN. In another embodiment, the gene is alsS. In another embodiment, the gene is fabF. In another embodiment, the gene is fabH. In another embodiment, the gene is pseudouridine synthase. In another embodiment, the gene is pyrG. In another embodiment, the gene is truA. In another embodiment, the gene is pabB. In another embodiment, the gene is an atp synthase gene (e.g. atpC, atpD-2, aptG, atpA-2, etc).

In another embodiment, the gene is phoP. In another embodiment, the gene is aroA and/or aroC. In another embodiment, the gene is aroD. In another embodiment, the gene is plcB.

In another embodiment, the Listeria strain is deficient in a peptide transporter. In another embodiment, the gene is ABC transporter/ATP-binding/permease protein. In another embodiment, the gene is oligopeptide ABC transporter/oligopeptide-binding protein. In another embodiment, the gene is oligopeptide ABC transporter/permease protein. In another embodiment, the gene is zinc ABC transporter/zinc-binding protein. In another embodiment, the gene is sugar ABC transporter. In another embodiment, the gene is phosphate transporter. In another embodiment, the gene is ZIP zinc transporter. In another embodiment, the gene is drug resistance transporter of the EmrB/QacA family. In another embodiment, the gene is sulfate transporter. In another embodiment, the gene is proton-dependent oligopeptide transporter. In another embodiment, the gene is magnesium transporter. In another embodiment, the gene is formate/nitrite transporter. In another embodiment, the gene is spermidine/putrescine ABC transporter. In another embodiment, the gene is Na/Pi-cotransporter. In another embodiment, the gene is sugar phosphate transporter. In another embodiment, the gene is glutamine ABC transporter. In another embodiment, the gene is major facilitator family transporter. In another embodiment, the gene is glycine betaine/L-proline ABC transporter. In another embodiment, the gene is molybdenum ABC transporter. In another embodiment, the gene is techoic acid ABC transporter. In another embodiment, the gene is cobalt ABC transporter. In another embodiment, the gene is ammonium transporter. In another embodiment, the gene is amino acid ABC transporter. In another embodiment, the gene is cell division ABC transporter. In another embodiment, the gene is manganese ABC transporter. In another embodiment, the gene is iron compound ABC transporter. In another embodiment, the gene is maltose/maltodextrin ABC transporter. In another embodiment, the gene is drug resistance transporter of the Bcr/CflA family. In another embodiment, the gene is a subunit of one of the above proteins.

In another embodiment, a recombinant Listeria strain of the present invention has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. Methods for passaging a recombinant Listeria strain through an animal host are well known in the art, and are described, for example, in U.S. patent application Ser. No. 10/541,614. Each possibility represents a separate embodiment of the present invention. Each Listeria strain and type thereof represents a separate embodiment of the present invention.

In another embodiment, the recombinant Listeria of methods and compositions of the present invention is stably transformed with a construct encoding an antigen or an LLO-antigen fusion. In one embodiment, the construct contains a polylinker to facilitate further subcloning. Several techniques for producing recombinant Listeria are known; each technique represents a separate embodiment of the present invention.

In another embodiment, the construct or heterologous gene is integrated into the Listerial chromosome using homologous recombination. Techniques for homologous recombination are well known in the art, and are described, for example, in Frankel, F R, Hegde, S, Lieberman, J, and Y Paterson. Induction of a cell-mediated immune response to HIV gag using Listeria monocytogenes as a live vaccine vector. J. Immunol. 155: 4766-4774. 1995; Mata, M, Yao, Z, Zubair, A, Syres, K and Y Paterson, Evaluation of a recombinant Listeria monocytogenes expressing an HIV protein that protects mice against viral challenge. Vaccine 19:1435-45, 2001; Boyer, J D, Robinson, T M, Maciag, P C, Peng, X, Johnson, R S, Paviakis, G, Lewis, M G, Shen, A, Siliciano, R, Brown, C R, Weiner, D, and Y Paterson. DNA prime Listeria boost induces a cellular immune response to SIV antigens in the Rhesus Macaque model that is capable of limited suppression of SIV239 viral replication. Virology. 333: 88-101, 2005. In another embodiment, homologous recombination is performed as described in U.S. Pat. No. 6,855,320. In another embodiment, a temperature sensitive plasmid is used to select the recombinants. Each technique represents a separate embodiment of the present invention.

In another embodiment, the construct or heterologous gene is integrated into the Listerial chromosome using transposon insertion. Techniques for transposon insertion are well known in the art, and are described, inter alia, by Sun et al. (Infection and Immunity 1990, 58: 3770-3778) in the construction of DP-L967. Transposon mutagenesis has the advantage, in another embodiment, that a stable genomic insertion mutant can be formed. In another embodiment, the position in the genome where the foreign gene has been inserted by transposon mutagenesis is unknown.

In another embodiment, the construct or heterologous gene is integrated into the Listerial chromosome using phage integration sites (Lauer P, Chow M Y et al, Construction, characterization, and use of two LM site-specific phage integration vectors. J Bacteriol 2002; 184(15): 4177-86). In another embodiment, an integrase gene and attachment site of a bacteriophage (e.g. U153 or PSA listeriophage) is used to insert the heterologous gene into the corresponding attachment site, which can be any appropriate site in the genome (e.g. comK or the 3′ end of the arg tRNA gene). In another embodiment, endogenous prophages are cured from the attachment site utilized prior to integration of the construct or heterologous gene. In another embodiment, this method results in single-copy integrants. Each possibility represents a separate embodiment of the present invention.

In another embodiment, the construct is carried by the Listeria strain on a plasmid. LM vectors that express antigen fusion proteins have been constructed via this technique. Lm-GG/E7 was made by complementing a prfA-deletion mutant with a plasmid containing a copy of the prfA gene and a copy of the E7 gene fused to a form of the LLO (hly) gene truncated to eliminate the hemolytic activity of the enzyme, as described herein. Functional LLO was maintained by the organism via the endogenous chromosomal copy of hly. In another embodiment, the plasmid contains an antibiotic resistance gene. In another embodiment, the plasmid contains a gene encoding a virulence factor that is lacking in the genome of the transformed Listeria strain. In another embodiment, the virulence factor is prfA. In another embodiment, the virulence factor is LLO. In another embodiment, the virulence factor is ActA. In another embodiment, the virulence factor is any of the genes enumerated above as targets for attenuation. In another embodiment, the virulence factor is any other virulence factor known in the art. Each possibility represents a separate embodiment of the present invention.

In another embodiment, a recombinant peptide of the present invention is fused to a Listerial protein, such as PI-PLC, or a construct encoding same. In another embodiment, a signal sequence of a secreted Listerial protein such as hemolysin, ActA, or phospholipases is fused to the antigen-encoding gene. In another embodiment, a signal sequence of the recombinant vaccine vector is used. In another embodiment, a signal sequence functional in the recombinant vaccine vector is used. Each possibility represents a separate embodiment of the present invention.

In another embodiment, the construct is contained in the Listeria strain in an episomal fashion. In another embodiment, the foreign antigen is expressed from a vector harbored by the recombinant Listeria strain. Each method of expression in Listeria represents a separate embodiment of the present invention.

In another embodiment, the present invention provides a method of inducing a cell-mediated immune response against an IgE protein in a subject, the method comprising the step of contacting the subject with an immunogenic composition comprising either (a) a recombinant peptide comprising the IgE protein or a fragment thereof; or (b) a nucleotide molecule encoding the recombinant peptide, thereby inducing a cell-mediated immune response against an IgE protein in a subject. In another embodiment, the cell-mediated immune response is a T cell response. In another embodiment, the IgE protein is endogenously expressed within the subject. Each possibility represents a separate embodiment of the present invention.

In another embodiment, the present invention provides a method of inducing a cell-mediated immune response against an IgE-expressing cell in a subject, the method comprising the step of contacting the subject with an immunogenic composition comprising either (a) a recombinant peptide comprising the IgE protein or a fragment thereof; or (b) a nucleotide molecule encoding the recombinant peptide, thereby inducing a cell-mediated immune response against an IgE-expressing cell in a subject. In another embodiment, the cell-mediated immune response is a T cell response. In another embodiment, the IgE protein is endogenously expressed within the subject. Each possibility represents a separate embodiment of the present invention.

As provided herein, vaccines of the present invention induce antigen-specific CTL. Thus, the vaccines are efficacious in eliminating cells containing antigens present in the vaccines, such as IgE and IgE fragments (e.g. those fragments enumerated herein). Further, CTL induced by vaccines of the present invention induce mucosal immunity, as evidenced by protection against viral infection at the mucosal surface of the lungs (Example 8).

As provided herein, methods for anti-IgE vaccination can be readily tested by determining serum IgE and IgG titers. Methods for determining serum IgE and IgG1 titers are well known in the art, and include 2-color ELISPOT assay, which can simultaneously detect distinct isotypes of antibody secreting cells (Czerkinsky et al., 1988). In another embodiment, measurement of IgG1 isotype responses serves as a specificity control to determine if treatment with the IgE recombinant vaccine affects only B cells secreting this isotype

In another embodiment of methods of the present invention, the subject is immunized with an immunogenic composition, vector, or recombinant peptide of the present invention. In another embodiment, the subject is administered the immunogenic composition, vector, or recombinant peptide. Each possibility represents a separate embodiment of the present invention.

In another embodiment, the present invention provides a method of treating, inhibiting, suppressing or ameliorating an allergy-induced asthma in a subject, comprising the step of contacting the subject with an immunogenic composition comprising either (a) a recombinant peptide comprising an IgE protein or a fragment thereof; or (b) a nucleotide molecule encoding the recombinant peptide, thereby treating, inhibiting, suppressing or ameliorating an allergy-induced asthma in a subject. In another embodiment, the IgE protein is endogenously expressed by the subject. Each possibility represents a separate embodiment of the present invention.

As provided herein, vaccines of the present invention are efficacious in eliminating cells containing newly synthesized IgE protein. Thus, vaccines of the present invention reduce systemic IgE levels, thereby significantly reducing the severity of, and in some cases eliminating, allergy and asthma.

In another embodiment, the present invention provides a method of treating, inhibiting, suppressing or ameliorating an allergy in a subject, comprising the step of contacting the subject with an immunogenic composition comprising either (a) a recombinant peptide comprising an IgE protein or a fragment thereof; or (b) a nucleotide molecule encoding the recombinant peptide, thereby treating, inhibiting, suppressing or ameliorating an allergy in a subject. In another embodiment, the IgE protein is endogenously expressed by the subject. Each possibility represents a separate embodiment of the present invention.

In another embodiment, a method of the present invention ameliorates allergy or asthma-associated episodic airflow obstruction. In another embodiment, a method of the present invention ameliorates allergy or asthma-associated inflammation of the airways. In another embodiment, a method of the present invention ameliorates allergy or asthma-associated enhanced bronchial reactivity (airways hyper-reactivity [AHR]) to inhaled spasmogenic stimuli.

In another embodiment, a method of the present invention ameliorates IgE production in response to accumulation of Th2 cell-containing inflammatory infiltrates in the lungs. In another embodiment, a method of the present invention ameliorates IgE production in response to a Th2 cytokine. In another embodiment, the cytokine is IL-4. In another embodiment, the cytokine is IL-13. In another embodiment, the cytokine is IL-5. In another embodiment, the cytokine is any other Th2 cytokine known in the art. Each possibility represents a separate embodiment of the present invention.

In another embodiment, a method of the present invention decreases activation of a cell or cell type that binds soluble IgE. In another embodiment, the cell type is mast cells. In another embodiment, the cell type is any other IgE-binding cell type known in the art. In another embodiment, the effect is mediated by a decrease in circulating IgE levels. In another embodiment, the effect is mediated by a decrease in lung IgE levels. Each possibility represents a separate embodiment of the present invention.

In another embodiment, a method of the present invention is used to treat AHR. In another embodiment, a method of the present invention is used to treat full-spectrum allergic disease. In another embodiment, a method of the present invention is used therapeutically. In another embodiment, a method of the present invention is used prophylactically. In another embodiment, the allergic disease comprises eosinophilia, IgE, IgG1, pulmonary Th2 cytokine responses, and/or AHR. In other embodiments, the present invention provides a method of treating any disease, disorder, symptom, or side effect associated with allergy or asthma. Each disease, disorder, and symptom represents a separate embodiment of the present invention. Each possibility represents a separate embodiment of the present invention.

In one embodiment, methods of the present invention are used to treat, suppress, inhibit, or prevent any of the above-described diseases, disorders, symptoms, or side effects associated with allergy or asthma. In one embodiment, “treating” refers to both therapeutic treatment and prophylactic or preventative measures, wherein the object is to prevent or lessen the targeted pathologic condition or disorder as described hereinabove. Thus, in one embodiment, treating may include directly affecting or curing, suppressing, inhibiting, preventing, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment, symptoms are primary, while in another embodiment, symptoms are secondary. In one embodiment, “primary” refers to a symptom that is a direct result of a particular disease or disorder, while in one embodiment, “secondary” refers to a symptom that is derived from or consequent to a primary cause. In one embodiment, the compounds for use in the present invention treat primary or secondary symptoms or secondary complications related to allergy or asthma. In another embodiment, “symptoms” may be any manifestation of a disease or pathological condition.

Thus, in one embodiment, the present invention provides a method of treating, preventing, inhibiting, and/or suppressing an allergy in a subject. In another embodiment, the present invention provides a method of treating, preventing, inhibiting, and/or suppressing allergy-induced asthma in a subject. In another embodiment, the present invention provides a method of treating, preventing, inhibiting, and/or suppressing an asthma episode in a subject. In another embodiment, the present invention provides a method of treating, preventing, inhibiting, and/or suppressing an IgE-mediated disease or disorder. In another embodiment, the present invention provides protection of a subject against asthma, allergy-induced asthma, an asthma episode, an IgE-mediated disease or disorder, or a combination thereof. In one embodiment, an IgE-mediated disease or disorder may comprise allergic disease, allergic asthma, hay fever, drug allergies, allergic bronchopulmonary aspergillosis (ABPA), pemphigus vulgaris, atopic dermatitis, or a combination thereof. In another embodiment, an IgE-mediated disease or disorder comprises urticaria, eczema conjunctivitis, rhinorrhea, rhinitis gastroenteritis, or a combination thereof. In another embodiment, an IgE-mediated disease or disorder comprises myeloma, multiple myeloma, Hodgkin's disease, Hyper-IgE syndrome, Wiskott-Aldrich syndrome, or a combination thereof.

In another embodiment, AHR, allergic lung disease [ALD] and allergic disease are measured as described herein. In another embodiment, another method known in the art is utilized. Methods for assessing AHR, ALD, and allergic disease are well known in the art, and are described, for example, in Schneider A M et al (Induction of pulmonary allergen-specific IgA responses or airway hyperresponsiveness in the absence of allergic lung disease following sensitization with limiting doses of ovalbumin-alum. Cell Immunol 2001 Sep. 15; 212(2):101-9) and Mattes J et al (IL-13 induces airways hyper-reactivity independently of the EL-4R alpha chain in the allergic lung. J Immunol 2001 Aug. 1; 167(3): 1683-92). Each method represents a separate embodiment of the present invention.

In another embodiment, the present invention provides a method of reducing an incidence of an asthma episode in a subject, comprising the step of contacting the subject with an immunogenic composition comprising either (a) a recombinant peptide comprising an IgE protein or a fragment thereof; or (b) a nucleotide molecule encoding the recombinant peptide, wherein the IgE protein is endogenously expressed by a cell of the subject, and wherein the immunogenic composition induces a formation of a T cell-mediated immune response against the IgE protein, thereby reducing an incidence of an asthma episode in a subject. In another embodiment, the recombinant peptide further comprises a non-IgE AA sequence. In another embodiment, the non-IgE AA sequence is any non-IgE AA sequence enumerated herein. Each possibility represents a separate embodiment of the present invention.

The T cell-mediated immune response induced by methods and compositions of the present invention comprises, in another embodiment, a CTL-mediated response. In another embodiment, the T cell involved in the T cell-mediated immune response is a CTL. In another embodiment, the immune response is a CD8⁺ T cell response. In another embodiment, the immune response is predominantly a CD8⁺ T cell response. Each possibility represents a separate embodiment of the present invention.

In another embodiment, the T cell-mediated immune response comprises a T helper cell. In another embodiment, the T cell involved in the T cell-mediated immune response is a T helper cell. In another embodiment, the immune response is a Th1-type response. In another embodiment, the immune response is a predominantly Th-1-type response. In another embodiment, the immune response is a predominantly cell-mediated, as opposed to antibody-mediated, response. Each possibility represents a separate embodiment of the present invention.

In another embodiment, an IgE-specific T cell induced by methods and compositions of the present invention is capable of lysing an IgE-producing B cell in the subject. In another embodiment, the IgE-specific T cell is capable of recognizing an IgE-producing B cell in the subject. In another embodiment, the T cell involved in the T cell-mediated immune response is capable of lysing an IgE-producing B cell in the subject. In another embodiment, the T cell is capable of recognizing an IgE-producing B cell in the subject. In another embodiment, the T cell lyses an IgE-producing B cell in the subject. In another embodiment, the T cell recognizes an IgE-producing B cell in the subject. In another embodiment, the T cell kills its target by a mechanism than CTL lysis. In another embodiment, the T cell kills its target by inducing apoptosis. In another embodiment, the T cell kills its target via FAS-FAS-ligand interaction. Each possibility represents a separate embodiment of the present invention.

The IgE-producing B cell that is recognized or lysed by a T cell induced by methods and compositions of the present invention produces, in another embodiment, a surface IgE receptor. In another embodiment, the IgE-producing B cell produces IgE antibody. In another embodiment, the IgE-producing B cell produces soluble IgE antibody. Each possibility represents a separate embodiment of the present invention.

In another embodiment, an IgE-specific T cell induced by methods and compositions of the present invention does not lyse a non-target cell that bears, but does not produce, IgE molecules. In another embodiment, the non-target cell is a mast cell. In another embodiment, the non-target cell is a basophil. In another embodiment, the non-target cell is a circulating basophil. In another embodiment, the non-target cell is an activated eosinophil. Each possibility represents a separate embodiment of the present invention.

In another embodiment, a method or immunogenic composition of methods and compositions of the present invention induces a cell-mediated immune response. In another embodiment, the immunogenic composition induces a predominantly cell-mediated immune response. In another embodiment, the immunogenic composition induces a predominantly Th1-type immune response. Each possibility represents a separate embodiment of the present invention.

The asthma that is treated by methods and compositions of the present invention is, in another embodiment, an allergy-induced asthma. In another embodiment, the asthma is an IgE-mediated asthma. In another embodiment, the asthma is any other type of asthma known in the art. Each possibility represents a separate embodiment of the present invention.

In another embodiment, the present invention provides a method of identifying a compound that ameliorates an IgE-mediated disease or disorder, the method comprising the steps of: (A) contacting a first animal with the compound, wherein the first animal has not been administered the recombinant peptide of claim 1 and wherein the first animal exhibits the IgE-mediated disease or disorder; (B) contacting a second animal with the compound, wherein the second animal has been administered the recombinant peptide of claim 1; and (C) measuring a clinical correlate of the IgE-mediated disease or disorder in the first animal and the second animal. In another embodiment, if the compound positively affects the clinical correlate in the first animal and does not affect the clinical correlate in the second animal, then the compound may be used to ameliorate the IgE-mediated disease or disorder.

In another embodiment, immune responses induced by methods and compositions of the present invention preferentially engender antigen specific CTL that recognize IgE fragments newly synthesized in the cytoplasm of the target cell. In another embodiment, these cells do not recognize cells that bear cytophilic IgE, such as mast cells or basophils. Each possibility represents a separate embodiment of the present invention.

In another embodiment, a vaccine or immunogenic composition of the present invention is administered alone to a subject. In another embodiment, the vaccine or immunogenic composition is administered together with another allergy or asthma therapy. Each possibility represents a separate embodiment of the present invention.

In another embodiment, the present invention provides a method of vaccinating a subject against an IgE-expressing tumor, neoplasia, or malignancy, comprising the step of performing a method of the present invention, thereby vaccinating a subject against an IgE-expressing tumor, neoplasia, or malignancy.

In another embodiment, the present invention provides a method of treating an IgE-expressing tumor, neoplasia, or malignancy, comprising the step of performing a method of the present invention, thereby treating an IgE-expressing tumor, neoplasia, or malignancy.

In another embodiment, the present invention provides a method of suppressing a formation of an IgE-expressing tumor, neoplasia, or malignancy, comprising the step of performing a method of the present invention, thereby suppressing a formation of an IgE-expressing tumor, neoplasia, or malignancy.

In other embodiments, the recombinant peptide, recombinant nucleic acid, IgE fragment, vaccine vector, or recombinant Listeria strain of any of the methods described above have any of the characteristics of a recombinant peptide, recombinant nucleic acid, IgE fragment, vaccine vector, or recombinant Listeria strain of compositions of the present invention. Each characteristic represents a separate embodiment of the present invention.

In another embodiment, a peptide of the present invention is homologous to a peptide enumerated herein. The terms “homology,” “homologous,” etc, when in reference to any protein or peptide, refer, in one embodiment, to a percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Methods and computer programs for the alignment are well known in the art.

Homology is, in another embodiment, determined by computer algorithm for sequence alignment, by methods well described in the art. For example, computer algorithm analysis of nucleic acid sequence homology can include the utilization of any number of software packages available, such as, for example, the BLAST, DOMAIN, BEAUTY (BLAST Enhanced Alignment Utility), GENPEPT and TREMBL packages.

In another embodiment, “homology” or “homologous” refers to identity to a non-IgE sequence selected from SEQ ID No: 1-14 of greater than 70%. In another embodiment, “homology” refers to identity to a sequence selected from SEQ ID No: 1-14 of greater than 72%. In another embodiment, “homology” refers to identity to one of SEQ ID No: 1-14 of greater than 75%. In another embodiment, “homology” refers to identity to a sequence selected from SEQ ID No: 1-14 of greater than 78%. In another embodiment, “homology” refers to identity to one of SEQ ID No: 1-14 of greater than 80%. In another embodiment, “homology” refers to identity to one of SEQ ID No: 1-14 of greater than 82%. In another embodiment, “homology” refers to identity to a sequence selected from SEQ ID No: 1-14 of greater than 83%. In another embodiment, “homology” refers to identity to one of SEQ ID No: 1-14 of greater than 85%. In another embodiment, “homology” refers to identity to one of SEQ ID No: 1-14 of greater than 87%. In another embodiment, “homology” refers to identity to a sequence selected from SEQ ID No: 1-14 of greater than 88%. In another embodiment, “homology” refers to identity to one of SEQ ID No: 1-14 of greater than 90%. In another embodiment, “homology” refers to identity to one of SEQ ID No: 1-14 of greater than 92%. In another embodiment, “homology” refers to identity to a sequence selected from SEQ ID No: 1-14 of greater than 93%. In another embodiment, “homology” refers to identity to one of SEQ ID No: 1-14 of greater than 95%. In another embodiment, “homology” refers to identity to a sequence selected from SEQ ID No: 1-14 of greater than 96%. In another embodiment, “homology” refers to identity to one of SEQ ID No: 1-14 of greater than 97%. In another embodiment, “homology” refers to identity to one of SEQ ID No: 1-14 of greater than 98%. In another embodiment, “homology” refers to identity to one of SEQ ID No: 1-14 of greater than 99%. In another embodiment, “homology” refers to identity to one of SEQ ID No: 1-14 of 100%. Each possibility represents a separate embodiment of the present invention.

In another embodiment, “homology” or “homologous” refers to identity to an IgE sequence selected from SEQ ID No: 15-19 of greater than 70%. In another embodiment, “homology” refers to identity to a sequence selected from SEQ ID No: 15-19 of greater than 72%. In another embodiment, “homology” refers to identity to one of SEQ ID No: 15-19 of greater than 75%. In another embodiment, “homology” refers to identity to a sequence selected from SEQ ID No: 15-19 of greater than 78%. In another embodiment, “homology” refers to identity to one of SEQ ID No: 15-19 of greater than 80%. In another embodiment, “homology” refers to identity to one of SEQ ID No: 15-19 of greater than 82%. In another embodiment, “homology” refers to identity to a sequence selected from SEQ ID No: 15-19 of greater than 83%. In another embodiment, “homology” refers to identity to one of SEQ ID No: 15-19 of greater than 85%. In another embodiment, “homology” refers to identity to one of SEQ ID No: 15-19 of greater than 87%. In another embodiment, “homology” refers to identity to a sequence selected from SEQ ID No: 15-19 of greater than 88%. In another embodiment, “homology” refers to identity to one of SEQ ID No: 15-19 of greater than 90%. In another embodiment, “homology” refers to identity to one of SEQ ID No: 15-19 of greater than 92%. In another embodiment, “homology” refers to identity to a sequence selected from SEQ ID No: 15-19 of greater than 93%. In another embodiment, “homology” refers to identity to one of SEQ ID No: 15-19 of greater than 95%. In another embodiment, “homology” refers to identity to a sequence selected from SEQ ID No: 15-19 of greater than 96%. In another embodiment, “homology” refers to identity to one of SEQ ID No: 15-19 of greater than 97%. In another embodiment, “homology” refers to identity to one of SEQ ID No: 15-19 of greater than 98%. In another embodiment, “homology” refers to identity to one of SEQ ID No: 15-19 of greater than 99%. In another embodiment, “homology” refers to identity to one of SEQ ID No: 15-19 of 100%. Each possibility represents a separate embodiment of the present invention.

In another embodiment, homology is determined via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y.). In other embodiments, methods of hybridization are carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA.

Protein and/or peptide homology for any AA sequence listed herein is determined, in another embodiment, by methods well described in the art, including immunoblot analysis, or via computer algorithm analysis of AA sequences, utilizing any of a number of software packages available, via established methods. Some of these packages include the FASTA, BLAST, MPsrch or Scanps packages, and, in another embodiment, employ the use of the Smith and Waterman algorithms, and/or global/local or BLOCKS alignments for analysis. Each method of determining homology represents a separate embodiment of the present invention.

In another embodiment of the present invention, “nucleic acids” or “nucleotide” refers to a string of at least two base-sugar-phosphate combinations. The term includes, in one embodiment, DNA and RNA. “Nucleotides” refers, in one embodiment, to the monomeric units of nucleic acid polymers. RNA is, in one embodiment, in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al, Genes & Devel 16: 2491-96 and references cited therein). DNA can be, in other embodiments, in form of plasmid DNA, viral DNA, linear DNA, or chromosomal DNA or derivatives of these groups. In addition, these forms of DNA and RNA can be single, double, triple, or quadruple stranded. The term also includes, in another embodiment, artificial nucleic acids that contain other types of backbones but the same bases. In one embodiment, the artificial nucleic acid is a PNA (peptide nucleic acid). PNA contain peptide backbones and nucleotide bases and are able to bind, in one embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothioate nucleic acids and PNA are known to those skilled in the art, and are described in, for example, Neilsen P E, Curr Opin Struct Biol 9:353-57; and Raz N K et al Biochem Biophys Res Commun. 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed. Each nucleic acid derivative represents a separate embodiment of the present invention.

In another embodiment, the present invention provides a kit comprising a compound or composition utilized in performing a method of the present invention. In another embodiment, the present invention provides a kit comprising a composition, tool, or instrument of the present invention. Each possibility represents a separate embodiment of the present invention.

### Pharmaceutical Compositions and Methods of Administration

“Pharmaceutical composition” refers, in another embodiment, to a therapeutically effective amount of the active ingredient, i.e. the recombinant peptide or vector comprising or encoding same, together with a pharmaceutically acceptable carrier or diluent. A “therapeutically effective amount” refers, in another embodiment, to that amount which provides a therapeutic effect for a given condition and administration regimen.

The pharmaceutical compositions containing the active ingredient can be, in another embodiment, administered to a subject by any method known to a person skilled in the art, such as parenterally, transmucosally, transdermally, intramuscularly, intravenously, intra-dermally, subcutaneously, intra-peritonealy, intra-ventricularly, intra-cranially, intra-vaginally, or intra-tumorally.

In another embodiment of methods and compositions of the present invention, the pharmaceutical compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound and the inert carrier or diluent, a hard gelating capsule.

In another embodiment, the pharmaceutical compositions are administered by intravenous, intra-arterial, or intra-muscular injection of a liquid preparation. Suitable liquid formulations include solutions, suspensions, dispersions, emulsions, oils and the like. In another embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In another embodiment, the pharmaceutical compositions are administered topically to body surfaces and are thus formulated in a form suitable for topical administration. Suitable topical formulations include gels, ointments, creams, lotions, drops and the like. For topical administration, the recombinant peptide or vector is prepared and applied as a solution, suspension, or emulsion in a physiologically acceptable diluent with or without a pharmaceutical carrier.

In another embodiment, the active ingredient is delivered in a vesicle, e.g. a liposome.

In other embodiments, carriers or diluents used in methods of the present invention include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

In other embodiments, pharmaceutically acceptable carriers for liquid formulations are aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs.

In another embodiment, parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

In another embodiment, the pharmaceutical compositions provided herein are controlled-release compositions, i.e. compositions in which the active ingredient is released over a period of time after administration. Controlled- or sustained-release compositions include formulation in lipophilic depots (e.g. fatty acids, waxes, oils). In another embodiment, the composition is an immediate-release composition, i.e. a composition in which all the active ingredient is released immediately after administration.

### EXPERIMENTAL DETAILS SECTION

### Example 1

### ActA-E7 and LLO-E7 Fusions Confer Anti-tumor Immunity Materials and Experimental Methods

### Construction of Lm-LLO-E7 and Lm-actA-E7

The Lm-LLO-E7 and Lm-ActA-E7 plasmids were created from pDP2028 (encoding LLO-NP), which was in turn created from pDP1659 as follows:

Plasmid pAM401, a shuttle vector able to replicate in both gram-negative and gram-positive bacteria, contains a gram-positive chloramphenicol resistance gene and gram negative tetracycline resistance determinant. To construct plasmid pDP1659, the DNA fragment encoding the first 420 AA of LLO and its promoter and upstream regulatory sequences was PCR amplified with LM genomic DNA used as a template and ligated into pUC19. PCR primers used were 5′-GGCCCGGGCCCCCTCCTTTGAT-3′ (SEQ ID No: 20) and 5′-GGTCTAGATCATAATTTACTTCATCC-3′ (SEQ ID No: 21). The DNA fragment encoding NP was similarly PCR amplified with linearized plasmid pAPR501 (obtained from Dr. Peter Palese, Mt. Sinai Medical School, New York), used as a template, and subsequently ligated as an in-frame translational fusion into pUC19 downstream of the hemolysin gene fragment. PCR primers used were 5′-GGTCTAGAGAATTCCAGCAAAAGCAG-3′ (SEQ ID No: 22) and 5′-GGGTCGACAAGGGTATTTTTCTTTAAT-3′ (SEQ ID No: 23). The fusion was then subcloned into the EcoRV and SalI sites of pAM401.

Plasmid pDP2028 was constructed by subcloning the prfA gene into the SalI site of pDP1659.

Lm-LLO-E7 (hly-E7 fusion gene in an episomal expression system; FIG. 1B) was created as follows: E7 was amplified by PCR using the primers 5′-GGCTCGAGCATGGAGATACACC-3′ (SEQ ID No: 24; XhoI site is underlined) and 5′-GGGGACTAGTTTATGGTTTCTGAGAACA-3′ (SEQ ID No: 25; SpeI site is underlined) and ligated into pCR2.1 (Invitrogen, San Diego, Calif.). E7 was excised from pCR2.1 by XhoI/SpeI digestion and ligated into pGG-55. The hly-E7 fusion gene and the pluripotential transcription factor prfA were cloned into pAM401, a multicopy shuttle plasmid (Wirth R et al, J Bacteriol, 165: 831, 1986), generating pGG-55. The hly promoter drives the expression of the first 441 AA of the hly gene product, (lacking the hemolytic C-terminus, having the sequence set forth in SEQ ID No: 2), which is joined by the XhoI site to the E7 gene, yielding a hly-E7 fusion gene that is transcribed and secreted as LLO-E7. Transformation of a prfA negative strain of Listeria, XFL-7 (provided by Dr. Hao Shen, University of Pennsylvania), with pGG-55 selected for the retention of the plasmid in vivo. The hly promoter and gene fragment were generated using primers 5′-GGGGGCTAGCCCTCCTTTGATTAGTATATTC-3′ (SEQ ID No: 26; NheI site is underlined) and 5′-CTCCCTCGAGATCATAATTTACTTCATC-3′ (SEQ ID No: 27; XhoI site is underlined). The prfA gene was PCR amplified using primers 5′-GACTACAAGGACGATGACCGACAAGTGATAACCCGGGATCTAAATAAATCCGTTT-3′ (SEQ ID No: 28; XbaI site is underlined) and 5′-CCCGTCGACCAGCTCTTCTTGGTGAAG-3′ (SEQ ID No: 29; SalI site is underlined).

Lm-E7 (single-copy E7 gene cassette integrated into Listeria genome; FIG. 1A) was generated by introducing an expression cassette containing the hly promoter and signal sequence driving the expression and secretion of E7 into the orfz domain of the LM genome. E7 was amplified by PCR using the primers 5′-GCGGATCCCATGGAGATACACCTAC-3′ (SEQ ID No: 30; BamHI site is underlined) and 5′-GCTCTAGATTATGGTTTCTGAG-3′ (SEQ ID No: 31; XbaI site is underlined). E7 was then ligated into the pZY-21 shuttle vector. LM strain 10403S was transformed with the resulting plasmid, pZY-21-E7, which includes an expression cassette inserted in the middle of a 1.6-kb sequence that corresponds to the orfX, Y, Z domain of the LM genome. The homology domain allows for insertion of the E7 gene cassette into the orfz domain by homologous recombination. Clones were screened for integration of the E7 gene cassette into the orfZ domain.

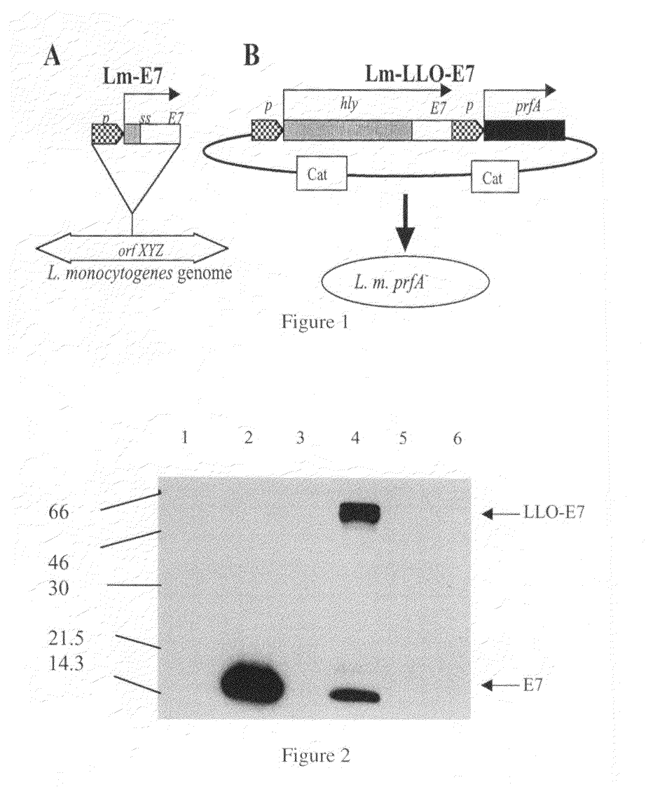

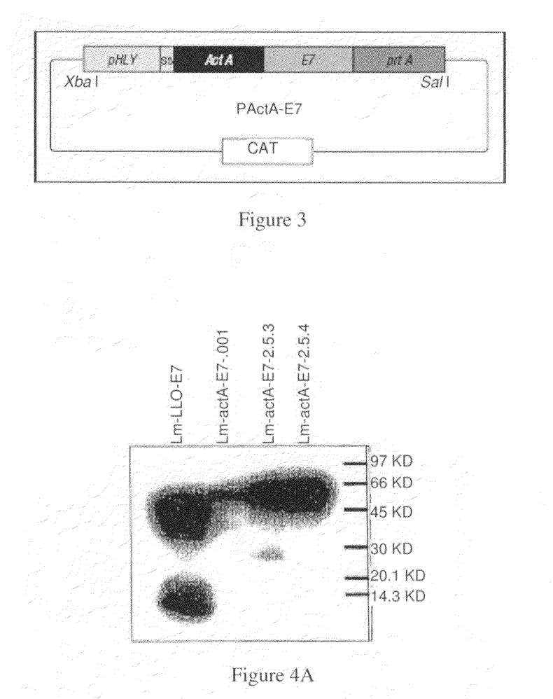

Bacteria were grown in brain heart infusion medium with (Lm-LLO-E7 and Lm-LLO-NP) or without (Lm-E7 and ZY-18) chloramphenicol (20 μg/ml), and were frozen in aliquots at −80° C. Expression was verified by Western blotting (FIG. 2).

Lm-actA-E7 was created from pDP-2028 (Lm-LLO-NP) as follows:

pDP-2028 is isogenic with Lm-LLO-E7, but expresses influenza antigen. Lm-actA-E7 contains a plasmid that expresses the E7 protein fused to a truncated version of the actA protein. Lm-actA-E7 was generated by introducing a plasmid vector pDD-1 constructed by modifying pDP-2028 into LM. pDD-1 comprises an expression cassette expressing a copy of the 310 bp hly promoter and the hly signal sequence (ss), which drives the expression and secretion of actA-E7; 1170 bp of the actA gene that comprises 4 PEST sequences (SEQ ID NO: 5) (the truncated ActA polypeptide consists of the first 390 AA of the molecule, SEQ ID NO: 4); the 300 bp HPV E7 gene; the 1019 bp prfA gene (controls expression of the virulence genes); and the CAT gene (chloramphenicol resistance gene) for selection of transformed bacteria clones. (FIG. 3) (Sewell et al. (2004), Arch. Otolaryngol. Head Neck Surg., 130: 92-97).

The hly promoter (pHly) and gene fragment (441 AA) were PCR amplified from pGG55 using primer 5′-GGGGTCTAGACCTCCTTTGATTAGTATATTC-3′ (Xba I site is underlined; SEQ ID NO: 32) and primer 5′-ATCTTCGCTATCTGTCGCCGCGGCGCGTGCTTCAGTTTGTTGCGC-′3 (Not I site is underlined. The first 18 nucleotides are the ActA gene overlap; SEQ ID NO: 33). The actA gene was PCR amplified from the LM 10403s wildtype genome using primer 5′-GCGCAACAAACTGAAGCAGCGGCCGCGGCGACAGATAGCGAAGAT-3′ (NotI site is underlined; SEQ ID NO: 34) and primer 5′-TGTAGGTGTATCTCCATGCTCGAGAGCTAGGCGATCAATTC-3′ (XhoI site is underlined; SEQ ID NO: 35). The E7 gene was PCR amplified from pGG55 using primer 5′-GGAATTGATCGCCTAGCTCTCGAGCATGGAGATACACCTACA-3′ (XhoI site is underlined; SEQ ID NO: 36) and primer 5′-AAACGGATTTATfTAGATCCCGGGTTATGGTTTCTGAGAACA-3′ (Xmal site is underlined; SEQ ID NO: 37). The prfA gene was PCR amplified from the LM 10403s wild-type genome using primer 5′-TGTTCTCAGAAACCATAACCCGGGATCTAAATAAATCCGTTT-3′ (XmaI site is underlined; SEQ ID NO: 38) and primer 5′-GGGGGTCGACCAGCTCTTCTTGGTGAAG-3′ (SalI site is underlined; SEQ ID NO: 39). The hly promoter-actA gene fusion (pHly-actA) was PCR generated and amplified from purified pHly and actA DNA using the upstream pHly primer (SEQ ID NO: 32) and downstream actA primer (SEQ ID NO: 35).

The E7 gene fused to the prfA gene (E7-prfA) was PCR generated and amplified from purified E7 and prfA DNA using the upstream E7 primer (SEQ ID NO: 36) and downstream prfA gene primer (SEQ ID NO: 39).

The pHly-actA fusion product fused to the E7-prfA fusion product was PCR generated and amplified from purified fused pHly-actA and E7-prfA DNA products using the upstream pHly primer (SEQ ID NO: 32) and downstream prfA gene primer (SEQ ID NO: 39) and ligated into pCRII (Invitrogen, La Jolla, Calif.). Competent E. coli (TOP10′F., Invitrogen, La Jolla, Calif.) were transformed with pCRII-ActAE7. After lysis and isolation, the plasmid was screened by restriction analysis using BamHI (expected fragment sizes 770 and 6400 bp) and BstXI (expected fragment sizes 2800 and 3900) and screened by PCR using the above-described upstream pHly primer and downstream prfA gene primer.

The pHly-ActA-E7-PrfA DNA insert was excised from pCRII by XbaI/SalI digestion with and ligated into Xba I/SalI digested pDP-2028. After transforming TOP10′F. competent E. coli (Invitrogen, La Jolla, Calif.) with expression system pActAE7, chloramphenicol resistant clones were screened by PCR analysis using the above-described upstream pHly primer and downstream prfA gene primer. A clone containing pActAE7 was amplified, and pActAE7 was isolated from the bacteria cell using a midiprep DNA purification system kit (Promega, Madison, Wis.). A prfA-negative strain of penicillin-treated Listeria (strain XFL-7) was transformed with expression system pActAE7, as described in Ikonomidis et al. (1994, J. Exp. Med. 180: 2209-2218) and clones were selected for the retention of the plasmid in vivo. Clones were grown in brain heart infusion medium (Difco, Detroit, Mich.) with 20 mcg (microgram)/ml (milliliter) chloramphenicol at 37° C. Bacteria were frozen in aliquots at −80° C.

### Immunoblot Verification of Antigen Expression

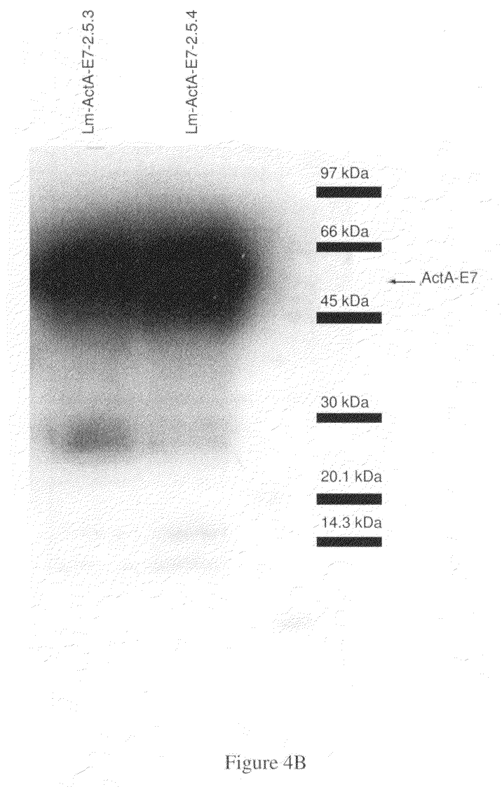

To verify that Lm-ActA-E7 secretes ActA-E7, (about 64 kD), Listeria strains were grown in Luria-Bertoni (LB) medium at 37° C. Protein was precipitated from the culture supernatant with trichloroacetic acid (TCA) and resuspended in 1× sample buffer with 0.1N sodium hydroxide. Identical amounts of each TCA-precipitated supernatant were loaded on 4% to 20% Tris-glycine sodium dodecyl sulfate-polyacrylamide gels (NOVEX, San Diego, Calif.). Gels were transferred to polyvinylidene difluoride membranes and probed with 1:2500 anti-E7 monoclonal antibody (Zymed Laboratories, South San Francisco, Calif.), then with 1:5000 horseradish peroxidase-conjugated anti-mouse IgG (Amersham Pharmacia Biotech, Little Chalfont, England). Blots were developed with Amersham enhanced chemiluminescence detection reagents and exposed to autoradiography film (Amersham) (FIG. 4).

### Tumor Regression Experiments

Six- to 8-wk-old C57BL/6 mice (Charles River) received 2×10⁵ TC-1 cells s.c. on the left flank. 1 week following tumor inoculation, the tumors had reached a palpable size of 4-5 mm in diameter. Mice were then treated on day 7 and 14 with 0.1 LD₅₀ of the Lm strains.

### Measurement of Tumor Growth

Tumors were measured every second day with calipers spanning the shortest and longest surface diameters. The mean of these two measurements was plotted as the mean tumor diameter in millimeters against various time points. Mice were sacrificed when the tumor diameter reached 20 mm. Tumor measurements for each time point are shown only for surviving mice.

### Results

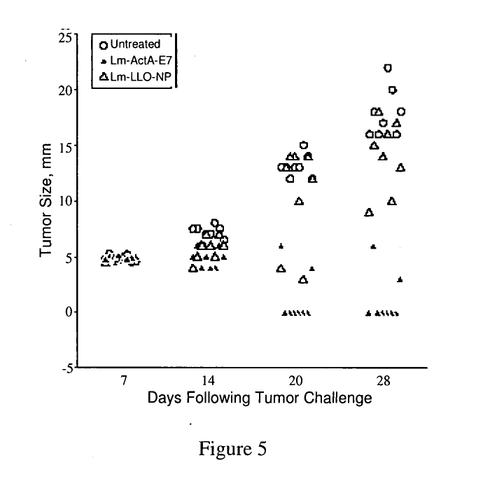

To determine the anti-tumor immunity induced by Listeria strains expressing the E7 antigen fused to ActA or to an LLO fragment (“Lm-ActA-E7” and “Lm-LLO-E7,” respectively), TC-1 tumor cells were implanted subcutaneously in mice and allowed to grow to a palpable size (approximately 5 millimeters [mm]). Mice were immunized i.p. with one LD₅₀ of either Lm-ActA-E7 (5×10⁸ CFU), Lm-LLO-E7 (10⁸ CFU) Lm-LLO-NP (additional negative control) or Lm-E7 (10⁶ CFU) on days 7 and 14. By day 26, all of the animals in the Lm-LLO-E7 and Lm-ActA-E7 were tumor free and remained so, whereas all of the naive animals and the animals immunized with Lm-LLO-NP or Lm-E7 grew large tumors (FIG. 5).

Thus, fusion to ActA, LLO, or fragments thereof confers increased immunogenicity upon antigens; specifically, cell-mediated immunogenicity.

### Example 2

### Fusion OF E7 to LLO or ActA Enhances E7-Specific Immunity and Generates Tumor-infiltrating E7-specific CD8⁺ Cells

### Materials and Experimental Methods

500 mcl of MATRIGEL®, containing 100 mcl phosphate buffered saline (PBS) with 2×10⁵ TC-1 tumor cells, plus 400 mcl of MATRIGEL® (BD Biosciences, Franklin Lakes, N.J.) were implanted subcutaneously on the left flank of 12 C57BL/6 mice (n=3). Mice were immunized intraperitoneally on day 7, 14 and 21, and spleens and tumors were harvested on day 28. Tumor MATRIGELs were removed from the mice and incubated at 4° C. overnight in tubes containing 2 ml RP 10 medium on ice. Tumors were minced with forceps, cut into 2 mm blocks, and incubated at 37° C. for 1 hour with 3 ml of enzyme mixture (0.2 mg/ml collagenase-P, 1 mg/ml DNAse-1 in PBS). The tissue suspension was filtered through nylon mesh and washed with 5% fetal bovine serum+0.05% of NaN₃ in PBS for tetramer and IFN-gamma staining.

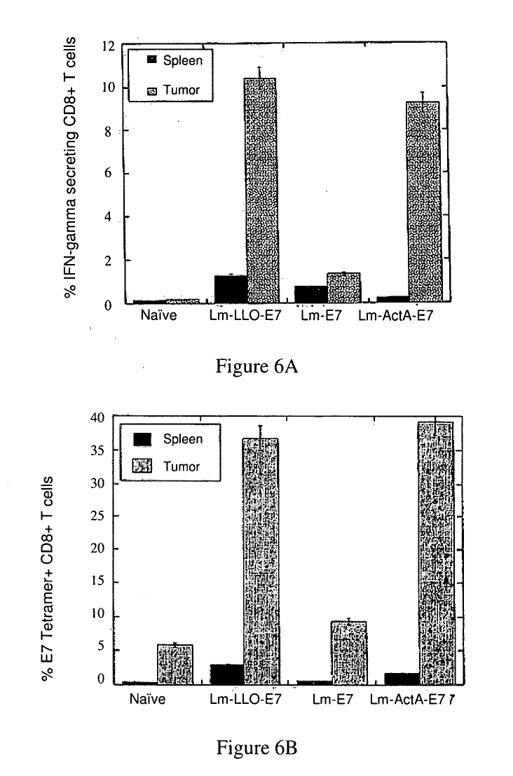

Splenocytes and tumor cells were incubated with 1 micromole (mcm) E7 peptide for 5 hours in the presence of brefeldin A at 10⁷ cells/ml. Cells were washed twice and incubated in 50 mcl of anti-mouse Fc receptor supernatant (2.4 G2) for 1 hour or overnight at 4° C. Cells were stained for surface molecules CD8 and CD62L, permeabilized, fixed using the permeabilization kit Golgi-stop® or Golgi-Plug® (Pharmingen, San Diego, Calif.), and stained for IFN-gamma. 500,000 events were acquired using two-laser flow cytometer FACSCalibur and analyzed using Cellquest Software (Becton Dickinson, Franklin Lakes, N.J.). Percentages of IFN-gamma secreting cells within the activated (CD62L^{low}) CD8⁺ T cells were calculated (FIG. 6 A).

For tetramer staining, H-2 D^{b} tetramer was loaded with phycoerythrin (PE)-conjugated E7 peptide (RAHYNIVTF, SEQ ID NO: 40), stained at rt for 1 hour, and stained with anti-allophycocyanin (APC) conjugated MEL-14 (CD62L) and FITC-conjugated CD8β at 4° C. for 30 min. Cells were analyzed comparing tetramer⁺CD8⁺ CD62L^{low} cells in the spleen and in the tumor (FIG. 6 B).

### Results

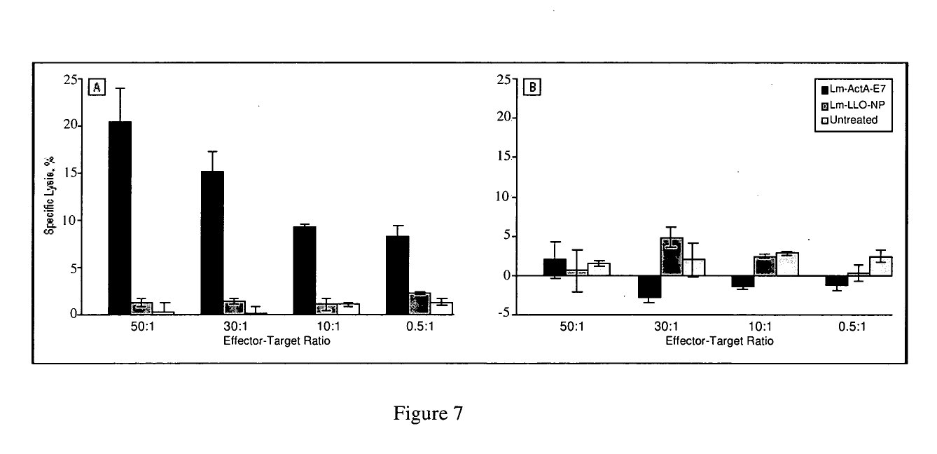

To analyze the ability of Lm-ActA-E7 to enhance antigen specific immunity, mice were implanted with TC-1 tumor cells and immunized with either Lm-LLO-E7 (1×10⁷ CFU), Lm-E7 (1×10⁶ CFU), or Lm-ActA-E7 (2×10⁸ CFU), or were untreated (naïve). Tumors of mice from the Lm-LLO-E7 and Lm-ActA-E7 groups contained a higher percentage of IFN-gamma-secreting CD8⁺ T cells (FIG. 6A) and tetramer-specific CD8⁺ cells (FIG. 6B) than in mice administered Lm-E7 or naive mice. In addition, Lm-ActA-E7 immunization induced E7-specific CTL activity (FIGS. 7A-B).

Thus, Lm-LLO-E7 and Lm-ActA-E7 are both efficacious at induction of tumor-infiltrating CD8⁺ T cells and tumor regression. Accordingly, LLO and ActA fusions are effective in methods and compositions of the present invention.

### Example 3

### Fusion to a Pest-like Sequence Enhances E7-specific Immunity

### Materials and Experimental Methods

### Constructs

Lm-PEST-E7, a Listeria strain identical to Lm-LLO-E7, except that it contains only the promoter and the first 50 AA of the LLO, was constructed as follows:

The hly promoter and PEST regions were fused to the full-length E7 gene by splicing by overlap extension (SOE) PCR. The E7 gene and the hly-PEST gene fragment were amplified from the plasmid pGG-55, which contains the first 441 amino acids of LLO, and spliced together by conventional PCR techniques. pVS16.5, the hly-PEST-E7 fragment and the LM transcription factor prfA were subcloned into the plasmid pAM401. The resultant plasmid was used to transform XFL-7, a prfA-negative strain of Listeria (provided by Dr. Jeffery Miller, University of California, Los Angeles), to create Lm-PEST-E7.

Lm-E7ₑₚᵢ is a recombinant strain that secretes E7 without the PEST region or an LLO fragment. The plasmid used to transform this strain contains a gene fragment of the hly promoter and signal sequence fused to the E7 gene. This construct differs from the original Lm-E7, which expressed a single copy of the E7 gene integrated into the chromosome. Lm-E7ₑₚᵢ is completely isogenic to Lm-LLO-E7 and Lm-PEST-E7, except for the form of the E7 antigen expressed.

Recombinant strains were grown in brain heart infusion medium with chloramphenicol (20 mcg/mL). Bacteria were frozen in aliquots at −80° C.

### Results

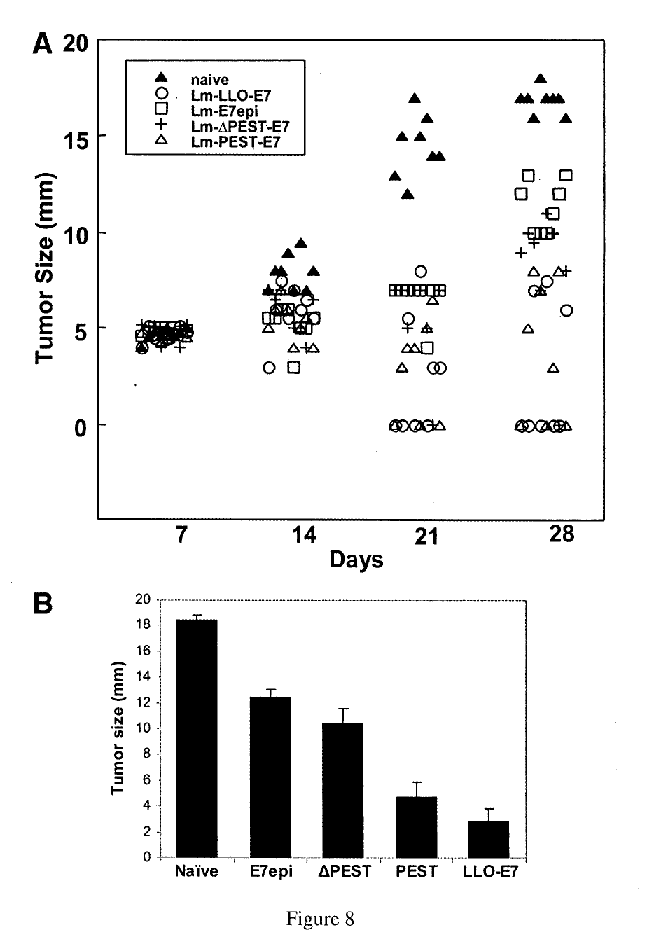

To test the effect on antigenicity of fusion to a PEST-like sequence, the LLO PEST-like sequence was fused to E7. Tumor regression studies were performed, as described for Example 1, in parallel with Listeria strain expressing LLO-E7 and E7 alone. Lm-LLO-E7 and Lm-PEST-E7 caused the regression 5/8 and 3/8 established tumors, respectively (FIG. 8A). In contrast, Lm-E7epi only caused tumor regression in 1/8 mice. A statistically significant difference in tumor sizes was observed between tumors treated with PEST-containing constructs (Lm-LLO-E7 or Lm-PEST-E7) and those treated with Lm-E7epi (Student's t test) (FIG. 8B).

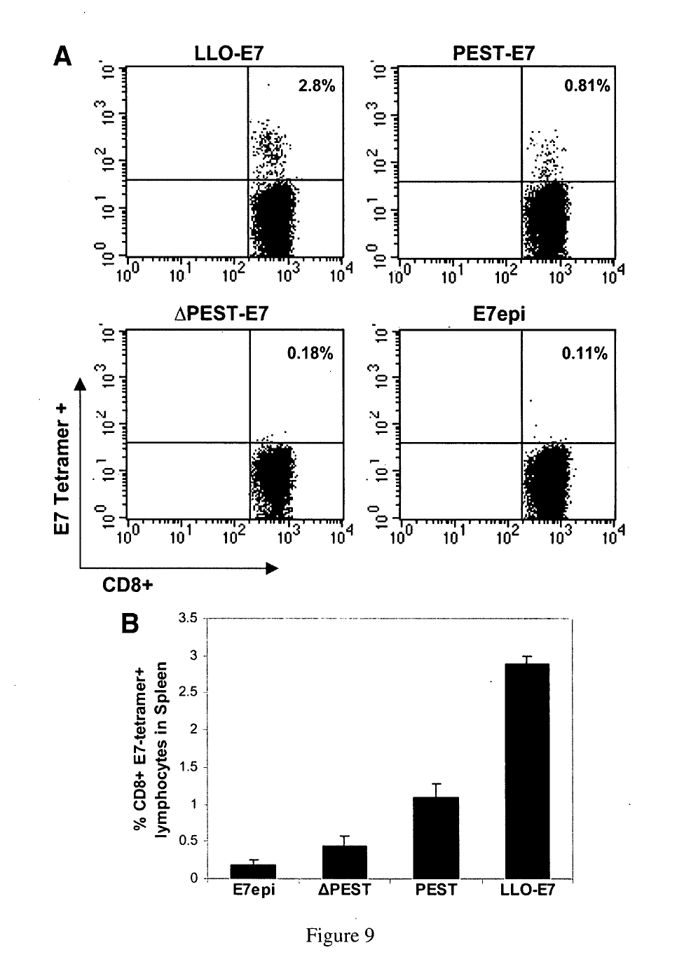

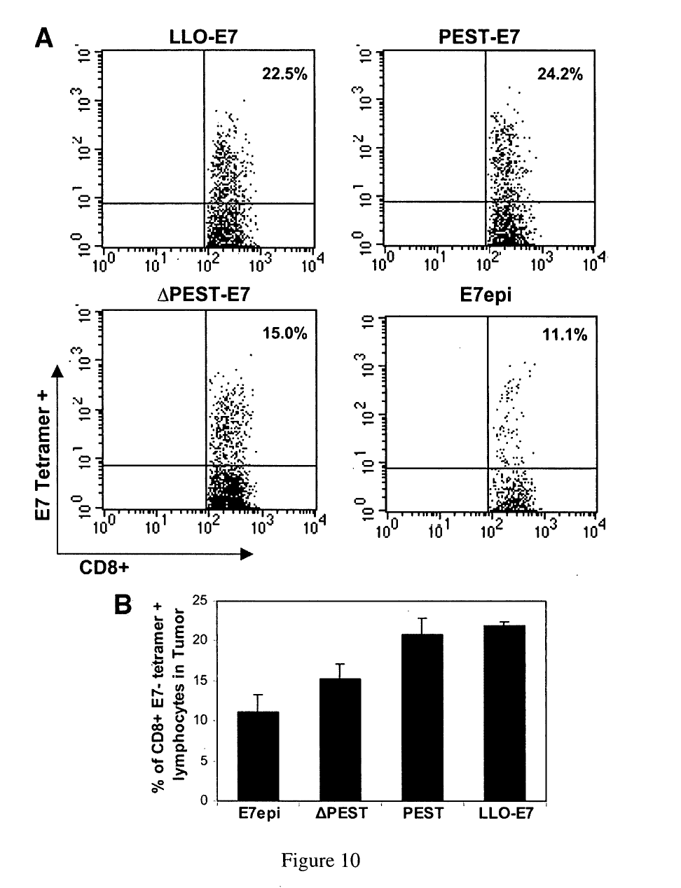

To compare the levels of E7-specific lymphocytes generated by the vaccines in the spleen, spleens were harvested on day 21 and stained with antibodies to CD62L, CD8, and the E7/Db tetramer. Lm-E7ₑₚᵢ induced low levels of E7 tetramer-positive activated CD8⁺ T cells in the spleen, while Lm-PEST-E7 and Lm-LLO-E7 induced 5 and 15 times more cells, respectively (FIG. 9A), a result that was reproducible over 3 separate experiments. Thus, fusion to PEST-like sequences increased induction of tetramer-positive splenocytes. The mean and SE of data obtained from the 3 experiments (FIG. 9B) demonstrate the significant increase in tetramer-positive CD8⁺ cells by Lm-LLO-E7 and Lm-PEST-E7 over Lm-E7epi (P<0.05 by Student's t test). Similarly, the number of tumor-infiltrating antigen-specific CD8⁺ T cells was higher in mice vaccinated with Lm-LLO-E7 and Lm-PEST-E7, reproducibly over 3 experiments (FIG. 10A-B). Average values of tetramer-positive CD8⁺ TILs were significantly higher for Lm-LLO-E7 than Lm-E7epi (P<0.05; Student's t test).

Thus, PEST-like sequences confer increased immunogenicity to antigens.

### Example 4

### Enhancement of Immunogenicity by Fusion of an Antigen to LLO does not Require a Listeria Vector

### Materials and Experimental Methods

### Construction of Vac-LLO-E7

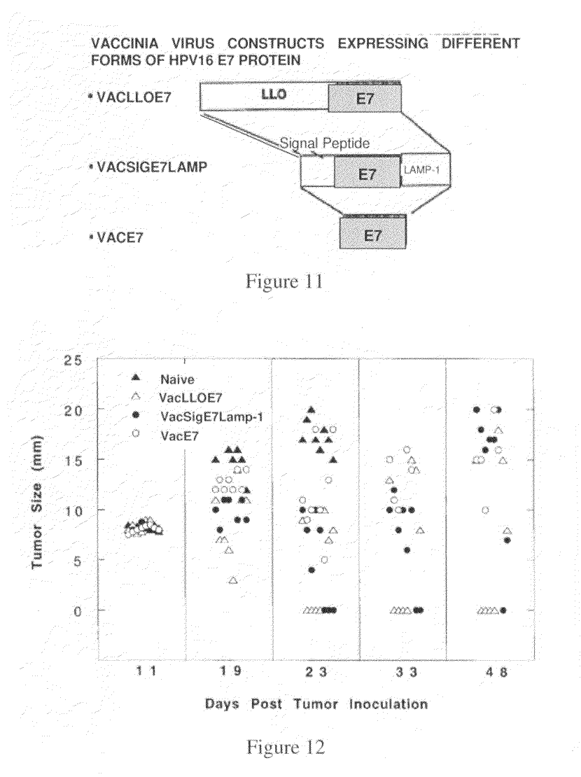

The WR strain of vaccinia was used as the recipient, and the fusion gene was excised from the Listerial plasmid and inserted into pSC11 under the control of the p75 promoter. This vector was chosen because it is the transfer vector used for the vaccinia constructs Vac-SigE7Lamp and Vac-E7 and therefore allowed direct comparison with Vac-LLO-E7. In this way all three vaccinia recombinants would be expressed under control of the same early/late compound promoter p7.5. In addition, SC11 allows the selection of recombinant viral plaques to TK selection and beta-galactosidase screening. FIG. 11 depicts the various vaccinia constructs used in these experiments. Vac-SigE7Lamp is a recombinant vaccinia virus that expressed the E7 protein fused between lysosomal associated membrane protein (LAMP-1) signal sequence and sequence from the cytoplasmic tail of LAMP-1.

The following modifications were made to allow expression of the gene product by vaccinia: (a) the T5XT sequence that prevents early transcription by vaccinia was removed from the 5′ portion of the LLO-E7 sequence by PCR; and (b) an additional XmaI restriction site was introduced by PCR to allow the final insertion of LLO-E7 into SC11. Successful introduction of these changes (without loss of the original sequence that encodes for LLO-E7) was verified by sequencing. The resultant pSCl 1-E7 construct was used to transfect the TK-ve cell line CV1 that had been infected with the wild-type vaccinia strain, WR. Cell lysates obtained from this co-infection/transfection step contain vaccinia recombinants that were plaque-purified 3 times. Expression of the LLO-E7 fusion product by plaque purified vaccinia was verified by Western blot using an antibody directed against the LLO protein sequence. In addition, the ability of Vac-LLO-E7 to produce CD8⁺ T cells specific to LLO and E7 was determined using the LLO (91-99) and E7 (49-57) epitopes of Balb/c and C57/BL6 mice, respectively. Results were confirmed in a chromium release assay.

### Results

To determine whether enhancement of immunogenicity by fusion of an antigen to LLO requires a Listeria vector, a vaccinia vector expressing E7 as a fusion protein with a non-hemolytic truncated form of LLO was constructed. Tumor rejection studies were performed with TC-1 as described for Example 1, but initiating treatment when the tumors were 3 mm in diameter (FIG. 12). By day 76, 50% of the Vac-LLO-E7 treated mice were tumor free, while only 25% of the Vac-SigE7Lamp mice were tumor free. In other experiments, LLO-antigen fusions were shown to be more immunogenic than E7 peptide mixed with SBAS2 or unmethylated CpG oligonucleotides in a side-by-side comparison.

These results show that (a) LLO-antigen fusions are immunogenic not only in the context of Listeria, but also in other contexts; and (b) the immunogenicity of LLO-antigen fusions compares favorably with other vaccine approaches known to be efficacious.

### Example 5

### LLO and ActA Fusions Overcome Immune Tolerance of E6/E7 Transgenic Mice to E7-expressing Tumors

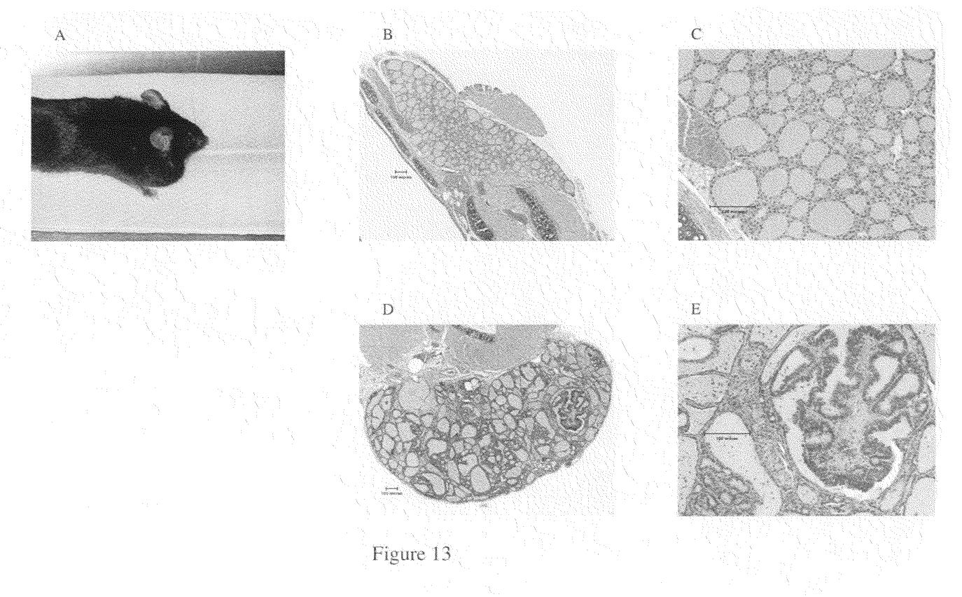

As a model of immune tolerance, E6/E7 transgenic mice were generated, and their phenotype assessed. The mice began to develop thyroid hyperplasia at 8 weeks and palpable goiters at 6 months. By 6 to 8 months, most mice exhibited thyroid cancer. Transgenic mice sacrificed at 6 months of age exhibited de-differentiation of the normal thyroid architecture, indicative of an early stage of cancer. The enlarged, de-differentiated cells were filled with colloid, where thyroid hormones accumulate (FIG. 13). Since E7 is a self antigen in these mice, the E6/E7 transgenic mice exhibited immune tolerance to E7.

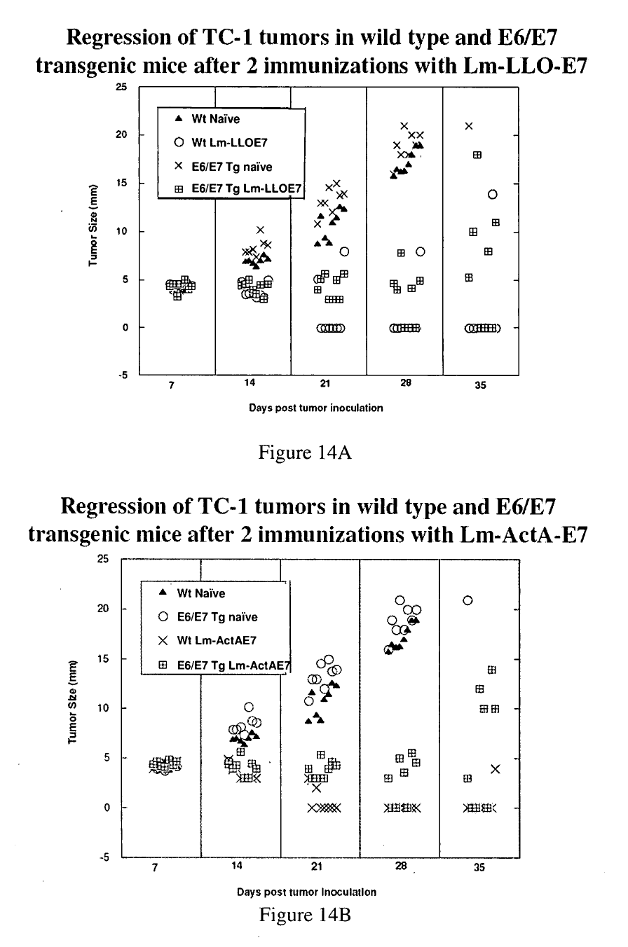

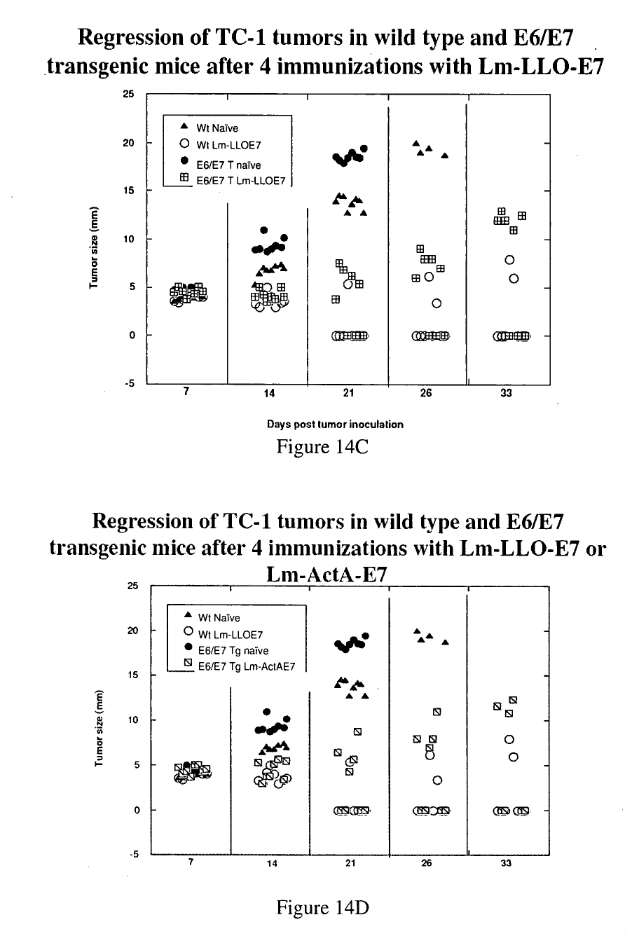

To examine the ability of vaccines of the present invention to overcome the immune tolerance of E6/E7 transgenic mice to E7-expressing tumors, 10⁵ TC-1 cells were implanted subcutaneously (s.c.) and allowed to form solid tumors in 6-8 week old wild-type and transgenic mice. Mice were left unimmunized (naïve) or were immunized 7 and 14 days later i.p. with LM-NP (control), 1×10⁸cfu LM-LLO-E7 (FIG. 14A) or 2.5×10⁸ cfu LM-ActA-E7 (FIG. 14B). The naïve mice had a large tumor burden, as anticipated, and were sacrificed by day 28 or 35 due to tumors of over 2 cm. By contrast, by day 35, administration of either LM-LLO-E7 or LM-ActA-E7 resulted in complete tumor regression in 7/8 or 6/8, respectively, of the wild-type mice and 3/8 of the transgenic mice. In the transgenic mice that did not exhibit complete tumor regression, a marked slowing of tumor growth was observed in the LM-LLO-E7-vaccinated and LM-ActA-E7-vaccinated mice.

In other experiments, additional vaccinations were administered on days 21 and 28. LM-LLO-E7 (FIG. 14C) or LM-ActA-E7 (FIG. 14D) induced complete tumor regression in 4/8 and 3/8 transgenic mice, respectively, and slowing of tumor growth in the remaining mice.

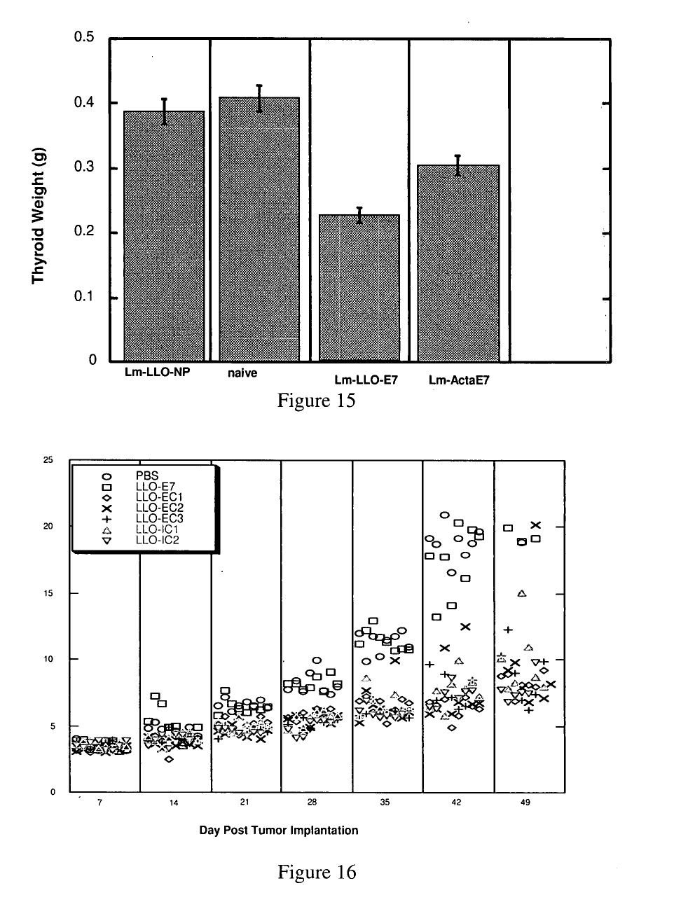

To investigate the ability of the vaccines to impact on autochthonous tumor growth, 6 to 8 week old mice were immunized with 1×10⁸ Lm-LLO-E7 or 2.5×10⁸ Lm-ActA-E7 once per month for 8 months. Mice were sacrificed 20 days after the last immunization and their thyroids removed and weighed. The results are shown as weight of thyroid for each vaccine group (FIG. 15).

The effectiveness of vaccines of the present invention in inducing complete tumor regression and/or slowing of tumor growth in transgenic mice was in marked contrast to the inefficacy of the peptide-based vaccine. Thus, vaccines of the present invention were able to overcome immune tolerance of E6/E7 transgenic mice to E7-expressing tumors.

### Example 6

### LLO-Her-2 Overcomes Immune Tolerance to a Self Antigen

### Materials and Experimental Methods

Rat Her-2/neu transgenic mice were purchased form Jackson laboratories and bred in the University of Pennsylvania vivarium. Young, virgin HER-2/neu transgenic mice that had not spontaneously developed tumors were injected with 5×10⁴ NT-2 cells. Because the transgenic mouse is profoundly tolerant to HER-2/neu, the minimum dose required for tumor growth in 100% of animals is much lower than wild-type mice (Reilly R T, Gottlieb M B et al, Cancer Res. 2000 Jul. 1; 60(13): 3569-76). NT-2 cells were injected into the subcutaneous space of the flank. Mice received 0.1 LD₅₀ of the Listeria vaccine on day 7 after tumor implantation (the time when 4-5 mm palpable tumors were detected) and weekly thereafter, for an additional 4 weeks.

### Results

The rat Her-2/neu gene differs from the mouse neu by 5-6% of AA residues, and thus is immunogenic in the mouse (Nagata Y, Furugen R et al, J. Immunol. 159: 1336-43). A transgenic mouse that over-expresses rat Her-2/neu under the transcriptional control of the Mouse Mammary Tumor Virus (MMTV) promoter and enhancer is immunologically tolerant to rat Her-2/neu. These mice spontaneously develop breast cancer. The MMTV promoter also operates in hematopoietic cells, rendering the mice profoundly tolerant to HER-2/neu. This, this mouse is a stringent model for human breast cancer and in general for tumors expressing antigens, such as Her-2/neu, that are expressed at low levels in normal tissue (Muller W. J. (1991) Expression of activated oncogenes in the murine mammary gland: transgenic models for human breast cancer. Canc Metastasis Rev 10: 217-27).

6-8 week-old HER-2/neu transgenic mice were injected with NT-2 cells, then immunized with each of the LM-ΔLLO-Her-2 vaccines, or with PBS or ΔLLO-E7 (negative controls). While most control mice had to be sacrificed by day 42 because of their tumor burden, tumor growth was controlled in all of the vaccinated mice (FIG. 16).

Thus, the ΔLM-LLO-Her-2 vaccines are able to break tolerance to self antigen expressed on a tumor cell, as evidenced by their ability to induce the regression of established NT-2 tumors. Accordingly, vaccines comprising LLO-antigen and ActA-antigen fusions are efficacious for breaking tolerance to self antigen with either Her-2 or E7, showing that findings of the present invention are generalizable and not specific to particular antigens.

### Example 7

### LLO-Her-2 Vaccines Control Spontaneous Tumor Growth in Her-2/Neu Transgenic Mice

### Materials and Experimental Methods

ΔLM-LLO-Her-2 vaccines were administered in the following amounts (cfu): Lm-LLO-EC1: 1×10⁷; Lm-Lm-LLO-EC2: 5×10⁷; LLO-EC3: 1×10⁸; Lm-LLO-IC2: 1×10⁷; Lm-LLO-IC1: 1×10⁷.

### Results

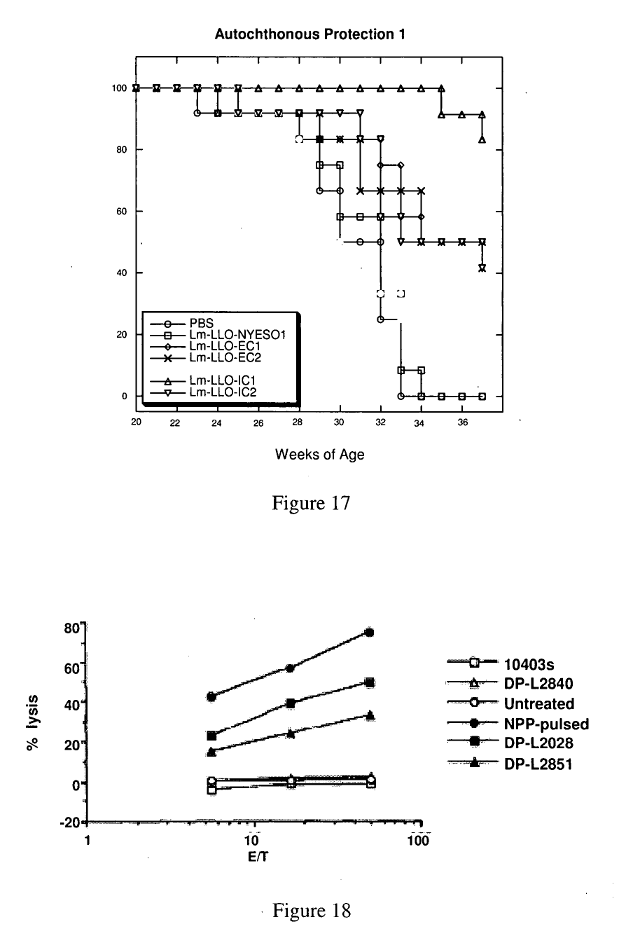

ΔLM-LLO-Her-2 vaccines were also evaluated for ability to prevent spontaneous tumor growth in the Her-2/neu transgenic mice. The transgenic mice (n=12 per vaccine group) were immunized 5 times with 0.1 LD₅₀ of one of the vaccine strains, beginning at age 6 weeks and continuing once every three weeks. Mice were monitored for tumor formation in the mammary glands. By week 35, all of the control mice (PBS or Lm-LLO-NY-ESO-1-immunized) had developed tumors. By contrast, 92% of the Lm-LLO-IC1 group were tumor free, as were 50% of the mice Lm-LLO-EC2, Lm-LLO-EC1, and Lm-LLO-IC2, and 25% of the mice immunized with Lm-LLO-EC3 (FIG. 17).

These findings confirm the results of the previous Examples, showing that vaccines of the present invention are able to break tolerance to self antigens and prevent spontaneous tumor growth.

### Example 8

### Mucosal Immune Responses are Induced by Listeria and LLO Fusion Vectors

### Materials and Experimental Methods

### Viruses

The influenza type A virus A/PR/8/34 belongs to the H1N1 subtype. The reassortment virus X31 (PR8×A/Aichi/68) differs from PR8 by expression of genes encoding H3 and N2, in place of H1N1, which are derived from the A/Aichi parent. Infectious virus stocks were grown in the allantoic cavity of 10 day old embryonated hen's eggs, and infectious allantoic fluid was stored in small aliquots at −70° C.

### Bacterial Strains and Growth Conditions

Plasmid pDP2028 was constructed as described in Example 1. Transformation of the prfA(−) strain DPL1075 with pDP2028 yielded strain DP-L2028, which secreted the fusion protein stably in vitro and in vivo.

Construction of strain DP-L2840. The splicing by overlap extenstion (SOE) PCR technique was used to replace the Kd restricted LLO epitope (residues 91-99) with the Kd restricted NP epitope, residues 147-155, and the modified hly gene was inserted into the PKSV7 temperature-sensitive vector to yield plasmid pDP2734. This plasmid was subsequently used to integrate the altered region into the bacterial chromosome.

Construction of strain DP-L2851. Plasmid pDP906 was derived by cloning a Sau96 fragment of the LM chromosome into pAM401. The chromosomal fragment codes for LLO and also includes the LLO promoter and the upstream regulatory sequences. No other complete open reading frames were present in this chromosomal fragment. Plasmid pDP906 was introduced into DP-L2840 by electroporation to yield DP-L2851. At every stage, engineering was verified by sequencing and restriction analysis.

### ⁵¹Cr Release Assays

Uninfected 5774 cells served as a negative control, and 5774 cells pulsed with the 147-158/R156-NP peptide as a positive control. P815 cells were labeled, pulsed with NP epitope peptide or control peptide, and used as targets at a density of 10⁴ cells per well (round-bottom 96-well plates, Costar). Alternatively, P815 cells were infected with influenza virus as follows: 10⁶ cells were pelleted and resuspended in 100 mcL of serum-free medium. 100 mcL of infectious allantoic fluid containing 1000 hemagglutinating units (HAU) of A/PR/8 virus were added, and cells rocked gently at 37° C. for 1 h. Subsequently, medium containing serum was added and cells were incubated overnight at 32° C. under 5% CO₂. The next day, infected cells were labeled with ⁵¹Cr and used as targets. Released ⁵¹Cr was determined on 100 mcL of supernatant. Specific lysis was calculated as 100×[(X−S)/(T−S)], where X=experimental counts per minute (c.p.m.), S=spontaneous c.p.m., and T=total (1% Triton-induced) c.p.m. Data shown are representative of several experiments with similar results.

### Determination of Viral Titers in the Lungs of Immunized Mice

Mice were immunized i.v. with either 0.1-0.2 LD₅₀ of the LM strains, 10⁷ pfu of the vaccinia strains (provided by Dr Jack Bennink, Laboratory of Viral Diseases, NIAID) or with 100 mcl of infectious allantoic fluid of X31 virus. Three weeks later, mice were inoculated intranasally (i.n.) with 50 mcL influenza A/PR/8 virus in PBS. The amount of virus given corresponded to 0.25 LD₅₀. Intranasal administration was performed under metofane-induced anesthesia. Mice were sacrificed after 5 days, and their lungs were removed and homogenized in serum-free (0.1% BSA) Iscove's medium. Viral titers in tenfold dilutions of lung extracts were determined as described.

### Results

Several NP-expressing Lm strains, all described above, were created. In the case of Lm-LLO-NP, NP was fused to an LLO fragment in the same manner as other constructs described above. In the case of DP-L2840, the Kd restricted NP epitope, which spans AA 147-155 of NP33, was incorporated into (i.e. embedded within) the secreted LLO molecule. Since flanking sequences have been shown to influence the efficiency of epitope processing, the AA residues within the K^{d} restricted LLO epitope GYKDGNEYI (residues 91-99; SEQ ID No: 41) were replaced with the residues from the K^{d} restricted epitope to ensure correct processing. The resulting strain DP-L2840 did not possess hemolytic activity, as determined by in vitro assays that measure lysis of sheep red blood cells, although it did secrete a mutant LLO molecule, as determined by Western blotting. The amount of LLO secreted by DP-L2840 was less than that precipitated from wild-type bacterial supernatants. To determine the effect of the difference in hemolytic activity, DP-L2840 was complemented in trans with a plasmid carrying a copy of the native hly gene, resulting in strain DP-L2851. DP-L2851 exhibited wild-type hemolytic activity on blood plates and grew more efficiently than DP-L2840 on a 5774 cell monolayer.

Cells infected with DP-L2028, but not DP-L2840, were able to present the NP epitope efficiently (FIG. 18). Cells infected with DP-L2851 were able to present the NP epitope, showing that the inability of DP-L2840 to present the NP epitope under the experimental conditions can be attributed to inefficient escape from the vacuole. The increased efficiency of DP-L2028 over DP-L2851 under the conditions utilized was likely due to the presence of a multicopy plasmid in DP-L2028, whereas DP-L2851 expresses the NP epitope from a single copy gene in the chromosome. Another possible explanation is the absence of CD4⁺ T cell epitopes in DP-L2028, which contains only the dominant CD8⁺ T cell epitope of NP.

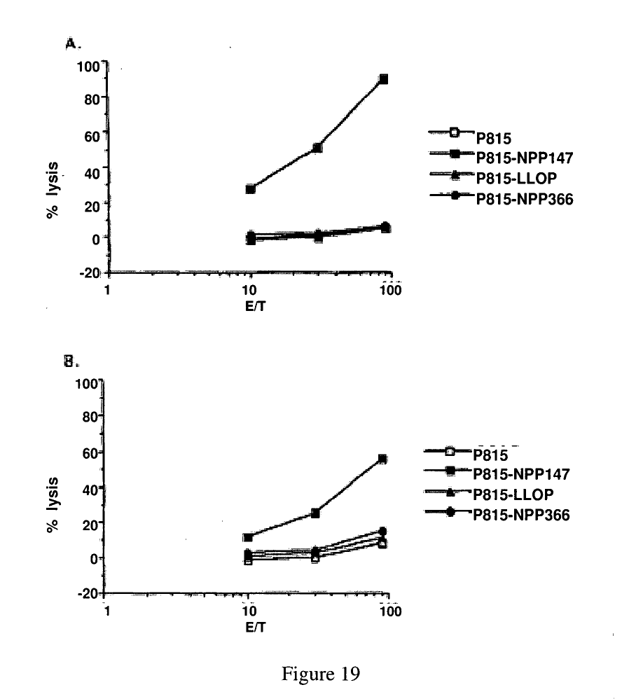

To determine the in vivo immunogenicity of DP-L2028 and DP-L2851, splenocytes were isolated from immunized BALB/c mice and stimulated in vitro with the K^{d}-restricted NP peptide. Both recombinant strains of LM were able to induce NP-specific CTL, as evidenced by cytolysis of peptide-pulsed and influenza-infected targets (FIG. 19).

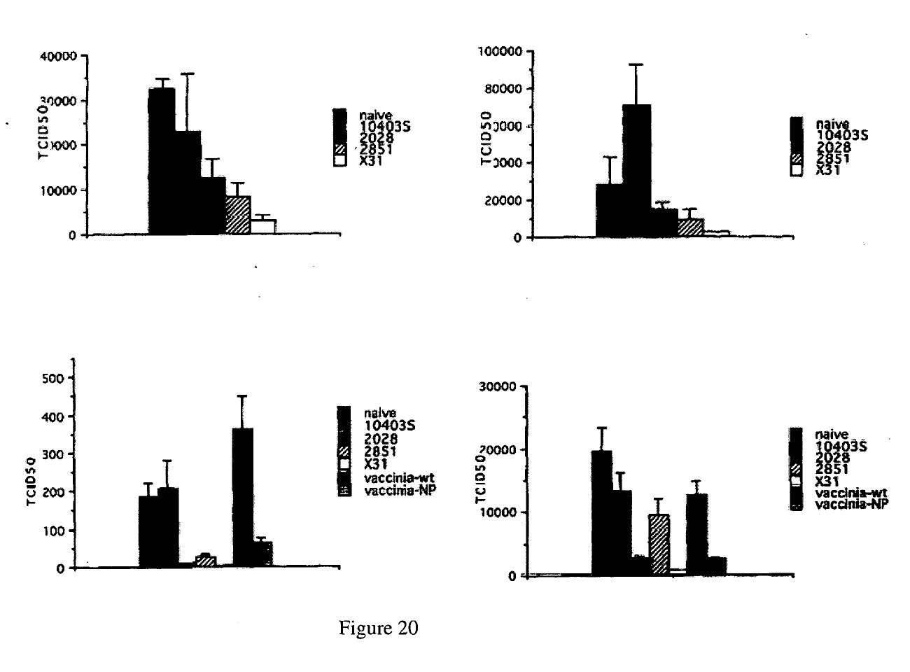

The protective effect of the vaccines was examined by challenging mice 3 weeks post-vaccination with a sublethal dose of A/PR/8/34 virus, from which the NP gene of the constructs was derived. Both DP-L2028 and DP-L2851 afforded statistically significant reductions (0.5-0.7 log) in the lung viral titers compared to naive mice or mice immunized with wild-type LM (FIG. 20 and Table 1).

TABLE 1
Reduction in lung virus titers (in log) in mice immunized with the indicated agents compared to naive
mice. Reductions for 4 experiments shown in FIG. 20, which used a total of 18 mice for 10403s,
DP-L2028, DP-L2851, and X31, and 12 mice for vaccinia-NP and vaccinia. *-reduction is significantly
different from 10403s (P < 0.05, Student's t-test).

| Immunizing | | | | | Mean +/− Standard |
|---|---|---|---|---|---|
| agent | Experiment 1 | Experiment 2 | Experiment 3 | Experiment 4 | Error |
| 10403S | −0.15 | 0.4 | −0.17 | 0.05 | 0.03 +/− −0.13 |
| DP-L2028 | −0.41 | −0.28 | −0.87 | −1.32 | −0.72 +/− −0.24* |
| DP-L2851 | −0.58 | −0.48 | −0.31 | −0.85 | −0.56 +/− −0.11* |
| X31 | −1.02 | −1.02 | −1.43 | −2.05 | −1.38 +/− −0.24* |
| vaccinia | ND | ND | −0.19 | 0.29 | 0.05 +/− −0.24 |
| vaccinia-NP | ND | ND | −0.89 | −0.47 | −0.68 +/− −0.21* |

*Reduction is significantly different from that conferred by 10403S (p < 0.05, Student's t test). ND = not done.

Thus, vaccines of the present invention induce cell-mediated immune responses against a variety of antigens. Further, the immune responses are induced whether the antigenic peptide is fused to or embedded within the LLO sequence, ActA sequence, or PEST-like sequence. Further, the immune responses confer protective immunity both systemically and in the mucosa.

### Example 9

### Construction of LM-IgE Vectors

Recombinant LM vaccine vectors are created, expressing and secreting into the host cell LLO or a fragment thereof fused to fragments of epsilon CH (specifically, the Cε1 domain [residues 134-224] and the complete Cε2 [residues 225-330], Cε3 [residues 331-437], and Cε4 [residues 438-547] and M1/M2. IgE CH and M1/M2 cDNA are generated using RT-PCR, with primers based on the murine cDNA sequence:

(SEQ ID NO: 16)

actgtgacctggtattcagactccctgaacatgagcactgtgaacttccc
tgccctcggttctgaactcaaggtcaccaccagccaagtgaccacagctg
gctaatggacgatcgggagataactgatacacttgcacaaactgttctaa
tcaaggaggaaggcaaactagcctctacctgcagtaaactcaacatcact
gagcagcaatggatgtctgaaagcaccttcacctgcaaggtcacctccca
aggcgtagactatttggcccacactcggagatgcccagatcaagcgagaa
gaatgtcaatgtgacgtggaaccaagagaagaagacttcagtctcagcat
cccagtggtacactaagcaccacaataacgccacaactagcatcaccaag
accccaggccagcgctcagcccccgaggtatatgtgttcccaccaccaga
ggaggagagcgaggacaaacgcacactcacctgtttgatccagaacttct
tccctgaggatatctctgtgcagtggctgggggatggcaaactgatctca
aacagccagcacagtaccacaacacccctgaaatccaatggctccaatca
aggcttcttcatcttcagtcgcctagaggtcgccaagacactctggacac
agagaaaacagttcacctgccaagtgatccatgaggcacttcagaaaccc
aggaaactggagaaaacaatatccacaagccttggtaacacctccctccg
tccctcctaggcctccatgtagctgtggtggggaaggtggatgacagaca
tccgctcactgttgtaacaccaggaagctaccccaataaacactcagtgc
ctg.

Secretion of each fusion protein into the bacterial growth media is verified by Western blot using an antibody to the LLO amino terminus (Singh et al, Fusion to Listeriolysin O and delivery by Listeria monocytogenes enhances the immunogenicity of HER-2/neu and reveals subdominant epitopes in the FVB/N mouse. J Immunol 2005; 175(6):3663-73).

Recombinant antigens are produced by subcloning the following genes into pGG-55. pGG-55 contains the necessary elements to produce about 10 micrograms/ml of secreted product in vitro:

1) For CH epsilon domains 1-4, the 2,4,6 TNP specific mouse hybridoma IGELa2 derived from BALB/c mice (H-2d) (GenBank Accession Numbers X65772 and X65774; SEQ ID No: 19) is utilized.

2) For the membrane exon of IgE, the B cell hybridoma IgE-53-569 (Bottcher et al, Production of monoclonal mouse IgE antibodies with DNP specificity by hybrid cell lines) is utilized.

### Example 10

### Generation of Specific Immune Responses Against IgE

### Constant Regions

Lm-LLO-E7 is included in all experiments below as a control to determine the extent of non-antigen-specific effects arising from the bacterial vector. To test the ability of the five Lm-LLO-CHε constructs to generate cell-mediated immunity in vivo to IgE constant regions, BALB/c are immunized mice parentally with LM vectors expressing LLO fused to IgE fragments. In other experiments, an oral route is utilized.

Anti-IgE humoral immune responses to the vaccines are determined by measuring production of serum antibodies by ELISA isotyping assay, and mucosal antibody response in orally inoculated mice. Minimal to undetectable humoral antibody responses are detected, consistent with previous experience with LM vectors and the intracellular life cycle of LM.

For anti-IgE cell-mediated immune responses, the following parameters are measured for lymphoid cells from immunized mice: 1) proliferation of CD4⁺ T cells upon stimulation with IgE; 2) secretion of cytokines, IFN-γ and IL-4 in response to IgE stimulation and verification of the phenotype of these cells by depleting either CD8- or CD4-positive cells; 3) generation of CTL that specifically recognize and lyse targets expressing IgE (e.g. IGELa2) or tumor target cells incubated with IgE-derived peptides. In additional experiments, cell phenotype, genetic restriction, and fine specificity of recognition of responses are determined.

One or more of the following antigen presenting cells (APC) is utilized for the in vitro expansion of IgE-specific CTL: Murine tumor cells such as P815 cells (an H-2d mastocytoma) and L cells transfected with individual H-2d MHC haplotypes, which are used to evaluate the MHC restriction of cloned CTL cells. The IgE heavy chain is introduced into the target cell by transfecting the line with the antigen cDNA, thereby synthesizing antigen in the cytosol. In other experiments, recombinant antigen is introduced into the cytoplasm by osmotic pinocytosis or antigenic peptides in the form of chemically homogenous synthetic peptides or protein digests. In additional experiments, peptides corresponding to two CTL epitopes for the BALB/c mouse in the CHε2 domain (positions 109 to 117 (LYCFIYGHI; SEQ ID No: 42; numbering begins with AA1 of the first constant region) and 113-121 (IYGHILNDV; SEQ ID No: 43) are synthesized, and immune responses thereto are assessed. Significant cell-mediated anti-IgE immune responses are observed, both to known CTL epitopes and to additional epitopes.

In other experiments, a single immunization is compared to multiple vaccines to optimize efficacy. In additional experiments, the time after immunization that CTL cells appear is determined. In additional experiments, fusions of an LLO sequence, ActA sequence, or PEST-like sequence to an antigen are tested in non-LM systems.

### Example 11

### Efficacy of Vaccines in Regulation and Suppression of Allergic Asthma

### Materials and Experimental Methods

Induction of Allergic Asthma in BALB/c Mice

Mice receive two i.p. injections of OVA-alum (2 mcg of OVA/mg alum, in 200 mcL saline) on days 0 and 14, followed by 1% OVA in saline aerosols on days 30, 32, and 34 (20 min/day). By day 35, mice exhibit significant airway eosinophilia and high levels of circulating. OVA-specific IgE antibodies mediated by a strong Th2 response in peripheral lymphoid organs and in the lungs. Mice are bled on day 35 for determination of IgE and IgG1 antibody titers and are assigned to experimental groups of equal extent of disease spread.

### Measurement of Allergic AHR

Allergic AHR is measured using the following techniques:

Lung inflammation; cellular and cytokine profile of bronchoalveolar lavage (BAL) fluid: Eosinophilic inflammatory infiltrate of the airways is a major pathological feature of asthma. To quantify the cellular changes, lungs are lavaged via the tracheal tube with 5 ml sterile saline, volume of collected bronchoalveolar (BAL) fluid per sample is measured, and leukocytes are counted (Coulter Counter, Coulter, Hialeah, Fl.). Differential cell counts are performed by counting at least 300 cells on cytocentrifuged preparations (Cytospin 2; Shandon, Runcorn, UK). Slides are stained with Leukostat (Fisher Diagnostics) and differentiated by standard hematological procedures. IL-2, IFN-γ, IL-4, IL-5, and Eotaxin levels are determined from cell free supernatants of BAL by ELISA, and total protein is determined by the standard method of Bradford.

Cytokine ELISAs: Cytokines are measured by sandwich ELISA following a standard protocol from Pharmingen (San Diego, Calif.).

Histopathology is performed in order to show concentration of inflammatory changes around the peribronchial and perivascular submucosal tissue. After lavage, lungs are inflated with 0.5 ml paraformaldehyde (4% w/Sodium Cacodylate, 0.1 M, pH 7.3) and fixed in the same solution for histological analysis. Inflation pressure is controlled in order to quantify the extent of emphysema in Surfactant protein D (SP-D)^{-/-} mice. For evaluation of airway inflammation, blocks of lung tissue are cut around the main bronchus and embedded in paraffin blocks. 5 mcm tissue sections are affixed to glass slides, and slides are deparaffinized, incubated in normal rabbit serum for 2 h at 37° C., stained with either rabbit anti-mouse MBP or normal rabbit preimmune control serum, and incubated overnight at 48° C. After washing and incubation in 1% chromotrope 2R (HARLECO, Gibbstown, N.J.) for 30 min, slides are placed in fluorescein-labeled goat anti-rabbit IgG for 30 min at 37° C., then examined with a Zeiss microscope equipped with a fluorescein filter system. The number of eosinophils in 0.06-mm² sections from the submucosal tissue around the major airways or peripheral (nonairway) tissue is analyzed with the IPLab2 software (Signal Analytics, Vienna, Va.)

Airway hyperresponsiveness to allergen challenge and to nonspecific stimuli such as metacholine (MCh) is also assessed. Lung resistance (RL) and dynamic compliance (Cdyn) is measured following intravenous administration of MCh as follows: Under anesthesia (100 mg/kg ketamine+20 mg/kg xylazine every 20 minutes before and during all surgical procedures), mice are administered 1.0 mg/kg pancuronium bromide, canulated, and ventilated (140 breaths/min; 0.2 ml tidal volume). Transduced alveolar pressure and airflow rate (Validyne DP45 and DP103, USA) is used to calculate lung resistance (RL) and dynamic compliance (Cdyn) by computer (Buxco Electronics, Inc. NY).

### Results

Mice injected with anti-IgD antiserum produce large amounts of IgE and IgG1 polyclonal antibody 8 days later. To determine the efficacy of vaccines of the present invention in regulation and suppression of allergic asthma, mice are immunized with Lm-LLO-CHε vaccines from the previous Example or with a control Lm vector. 8 days following injection with 200-300 mcg of anti-IgD, IgE and IgG1 serum titers are determined by ELISA, and IgE- and IgG1-secreting cells in the spleens are quantified by ELISPOT. This experiment is repeated at varying time intervals after vaccine administration in order to determine induction of long-term immunological memory. In other experiments, effects of anti-IgE vaccines on a secondary IgE antibody response are assessed in a mouse model of AHR.

To determine the effect of vaccines of the present invention on allergic asthma, asthma is induced, and mice are subsequently vaccinated with Lm-LLO-CHε or Lm-LLO-E7 (antigen control). Anti-OVA IgE antibodies and cells secreting same, but not IgG antibodies, are suppressed in the experimental group. Additional mice are sacrificed 1 week after vaccination and at later time points, and lungs and spleens are removed for assessment of Th2 responses by measuring levels of IL-4, IL-5, IL-9 and IL-13 and IFN-γ.

To determine the impact of vaccines of the present invention on airway hyper-responsiveness, asthmatic mice are vaccinated with anti-IgE or control vaccines. Two weeks later (a rest period to allow the asthma to wane), mice are challenged with increasing doses of methacholine and their AHR measured as described above.

To determine the role of CTL in the above effects, CD8⁺ T cells are prepared from the spleens of BALB/c mice immunized with anti-IgE and control vaccines. Cells are adoptively transferred to syngeneic mice at varying time periods prior to exposure to OVA aerosols on day 30 and onwards, and immune parameters are assayed as described above.

What is claimed is:

1. A recombinant peptide comprising a fragment of an IgE constant region, and a non-IgE amino acid sequence selected from a non-hemolytic listeriolysin (LLO) amino acid sequence, an ActA amino acid sequence, or a PEST-like amino acid sequence.

2. The recombinant peptide of claim 1, made by a process comprising the step of translation of a nucleotide molecule encoding said recombinant polypeptide.

3. The recombinant peptide of claim 1, made by a process comprising the step of chemically conjugating a polypeptide comprising said fragment of an IgE constant region to a polypeptide comprising said non-IgE amino acid sequence

4. The recombinant peptide of claim 1, wherein said IgE constant region is selected from a C epsilon-1 domain, a C epsilon-2 domain, a C epsilon-3 domain, a C epsilon-4 domain, an M1 domain, a M2 domain, and an M1/M2 domain.

5. The recombinant peptide of claim 1, wherein said fragment of an IgE constant region is fused to said non-IgE amino acid sequence.

6. The recombinant peptide of claim 1, wherein said fragment of an IgE constant region is embedded within said non-IgE amino acid sequence.

7. A vaccine comprising the recombinant polypeptide of claim 1 and an adjuvant.

8. A recombinant vaccine vector encoding the recombinant polypeptide of claim 1.

9. A recombinant Listeria strain comprising the recombinant polypeptide of claim 1.

10. The recombinant Listeria strain of claim 8, wherein said recombinant Listeria strain is a recombinant Listeria monocytogenes strain.

11. The recombinant Listeria strain of claim 8, wherein said recombinant Listeria strain has been passaged through an animal host.

12. A nucleotide molecule encoding the recombinant polypeptide of claim 1.

13. A vaccine comprising the nucleotide molecule of claim 12 and an adjuvant.

14. A recombinant vaccine vector comprising the nucleotide molecule of claim 12.

15. A recombinant Listeria strain comprising the nucleotide molecule of claim 12.

16. The recombinant Listeria strain of claim 15, wherein said recombinant Listeria strain is a recombinant Listeria monocytogenes strain.

17. The recombinant Listeria strain of claim 15, wherein said recombinant Listeria strain has been passaged through an animal host.

18. A recombinant Listeria strain expressing a peptide, said peptide comprising a fragment of an IgE constant region.

19. The recombinant Listeria strain of claim 18, wherein said peptide further comprises a non-IgE amino acid sequence.

20. The recombinant Listeria strain of claim 18, wherein said non-IgE amino acid sequence is selected from a non-hemolytic listeriolysin (LLO) amino acid sequence, an ActA amino acid sequence, and a PEST-like amino acid sequence.

21. The recombinant Listeria strain of claim 18, wherein said IgE constant region is selected from a C epsilon-1 domain, a C epsilon-2 domain, a C epsilon-3 domain, a C epsilon-4 domain, an M1 domain, a M2 domain, and an M1/M2 domain.

22. A vaccine comprising the recombinant Listeria strain of claim 18 and an adjuvant.

23. A method of inducing a cell-mediated immune response against an IgE protein in a subject, wherein said IgE protein is endogenously expressed by a cell of said subject, the method comprising contacting said subject with an immunogenic composition comprising either:
(a) a recombinant peptide comprising said IgE protein or a fragment thereof; or
(b) a nucleotide molecule encoding said recombinant peptide, wherein said immunogenic composition comprises an adjuvant that favors a predominantly Th1-type immune response, thereby inducing a cell-mediated immune response against an IgE protein in a subject.

24. The method of claim 23, wherein said immunogenic composition comprises a recombinant vaccine vector.

25. The method of claim 23, wherein said recombinant peptide further comprises a non-IgE amino acid sequence.

26. The method of claim 23, wherein said non-IgE amino acid sequence is selected from a non-hemolytic listeriolysin (LLO) amino acid sequence, an ActA amino acid sequence, and a PEST-like amino acid sequence.

27. A method of treating, inhibiting, suppressing or ameliorating an allergy in a subject, comprising the step of contacting said subject with an immunogenic composition comprising either (a) a recombinant peptide comprising an IgE protein or a fragment thereof; or (b) a nucleotide molecule encoding said recombinant peptide, wherein said IgE protein is endogenously expressed by a cell of said subject, and wherein said immunogenic composition induces a formation of a T cell-mediated immune response against said IgE protein, thereby of treating, inhibiting, suppressing or ameliorating an allergy in a subject.

28. The method of claim 27, wherein said immunogenic composition comprises a recombinant vaccine vector.

29. The method of claim 27, wherein said recombinant peptide further comprises a non-IgE amino acid sequence.

30. The method of claim 29, wherein said non-IgE amino acid sequence is selected from a non-hemolytic listeriolysin (LLO) amino acid sequence, an ActA amino acid sequence, and a PEST-like amino acid sequence.

31. The method of claim 27, wherein said T cell is a cytotoxic T lymphocyte.

32. The method of claim 27, wherein said T cell is a T helper cell.

33. The method of claim 27, wherein said T cell is capable of lysing an IgE-producing B cell in said subject.

34. A method of treating, inhibiting, suppressing, or ameliorating an allergy-induced asthma in a subject, comprising the step of contacting said subject with an immunogenic composition comprising either (a) a recombinant peptide comprising an IgE protein or a fragment thereof; or (b) a nucleotide molecule encoding said recombinant peptide, wherein said IgE protein is endogenously expressed by a cell of said subject, and wherein said immunogenic composition induces a formation of a T cell-mediated immune response against said IgE protein, thereby of treating, inhibiting, suppressing or ameliorating an allergy-induced asthma in a subject.

35. The method of claim 34, wherein said immunogenic composition comprises a recombinant vaccine vector.

36. The method of claim 34, wherein said recombinant peptide further comprises a non-IgE amino acid sequence.

37. The method of claim 36, wherein said non-IgE amino acid sequence is selected from a non-hemolytic listeriolysin (LLO) amino acid sequence, an ActA amino acid sequence, and a PEST-like amino acid sequence.

38. The method of claim 34, wherein said T cell is a cytotoxic T lymphocyte.

39. The method of claim 34, wherein said T cell is a T helper cell.

40. The method of claim 34, wherein said T cell is capable of lysing an IgE-producing B cell in said subject.

41. A method of reducing an incidence of an asthma episode in a subject, comprising the step of contacting said subject with an immunogenic composition comprising either (a) a recombinant peptide comprising an IgE protein or a fragment thereof; or (b) a nucleotide molecule encoding said recombinant peptide, wherein said IgE protein is endogenously expressed by a cell of said subject, and wherein said immunogenic composition induces a formation of a T cell-mediated immune response against said IgE protein, thereby reducing an incidence of an asthma episode in a subject.

42. The method of claim 41, wherein said immunogenic composition comprises a recombinant vaccine vector.

43. The method of claim 41, wherein said recombinant peptide further comprises a non-IgE amino acid sequence.

44. The method of claim 43, wherein said non-IgE amino acid sequence is selected from a non-hemolytic listeriolysin (LLO) amino acid sequence, an ActA amino acid sequence, and a PEST-like amino acid sequence.

45. The method of claim 41, wherein said T cell is a cytotoxic T lymphocyte.

46. The method of claim 41, wherein said T cell is a T helper cell.

47. The method of claim 41, wherein said T cell is capable of lysing an IgE-producing B cell in said subject.

48. The method of claim 41, wherein said asthma is an allergy-induced asthma.

49. A method of treating, inhibiting, suppressing, or ameliorating an IgE-mediated disease or disorder in a subject, comprising the step of contacting said subject with an immunogenic composition comprising either (a) a recombinant peptide comprising an IgE protein or a fragment thereof; or (b) a nucleotide molecule encoding said recombinant peptide, wherein said IgE protein is endogenously expressed by a cell of said subject, and wherein said immunogenic composition induces a formation of a T cell-mediated immune response against said IgE protein, thereby treating, inhibiting, suppressing, or ameliorating an IgE-mediated disease or disorder in a subject.

50. The method of claim 49, wherein said immunogenic composition comprises a recombinant vaccine vector.

51. The method of claim 49, wherein said recombinant peptide further comprises a non-IgE amino acid sequence.

52. The method of claim 51, wherein said non-IgE amino acid sequence is selected from a non-hemolytic listeriolysin (LLO) amino acid sequence, an ActA amino acid sequence, and a PEST-like amino acid sequence.

53. The method of claim 49, wherein said T cell is a cytotoxic T lymphocyte.

54. The method of claim 49, wherein said T cell is a T helper cell.

55. The method of claim 49, wherein said T cell is capable of lysing an IgE-producing B cell in said subject.

56. The method of claim 49, wherein said IgE mediated disease or disorder comprises asthma.

57. The method of claim 49, wherein said IgE mediated disease or disorder comprises allergy-induced asthma.

58. The method of claim 49, wherein said IgE mediated disease or disorder comprises hay fever.

59. The method of claim 49, wherein said IgE mediated disease or disorder comprises drug allergies.

60. The method of claim 49, wherein said IgE mediated disease or disorder comprises pemphigus vulgaris.

61. The method of claim 49, wherein said IgE mediated disease or disorder comprises atopic dermatitis.

62. The method of claim 49, wherein said IgE mediated disease or disorder comprises urticaria, eczema conjunctivitis, rhinorrhea, rhinitis gastroenteritis, or a combination thereof.

63. The method of claim 49, wherein said IgE mediated disease or disorder comprises myeloma, Hodgkin's disease, Hyper-IgE syndrome, Wiskott-Aldrich syndrome, or a combination thereof.

64. A method of identifying a compound that ameliorates an IgE-mediated disease or disorder, the method comprising the steps of:
A. contacting a first animal with said compound, wherein said first animal has not been administered the recombinant peptide of claim 1 and wherein said first animal exhibits said IgE-mediated disease or disorder;
B. contacting a second animal with said compound, wherein said first animal has been administered the recombinant peptide of claim 1; and
C. measuring a clinical correlate of said IgE-mediated disease or disorder in said first animal and said second animal;
whereby, if said compound positively affects said clinical correlate in said first animal and does not affect said clinical correlate in said second animal, then said compound ameliorates said IgE-mediated disease or disorder.


SEQUENCE LISTING (44 sequences)

SEQ ID NO: 1 - protein, 529 aa, Listeria monocytogenes
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 2 - protein, 441 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 3 - protein, 416 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 4 - protein, 390 aa, Listeria monocytogenes
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 5 - protein, 1170 aa, Listeria monocytogenes
ATGCGTGCGATGATGGTGGTTTTCATTACTGCCAATTGCATTACGATTAACCCCGACATAATATTTGCAGCGACAGATAGCGAAGATTCTAGTCTAAACACAGATGAATGGGAAGAAGAAAAAACAGAAGAGCAACCAAGCGAGGTAAATACGGGACCAAGATACGAAACTGCACGTGAAGTAAGTTCACGTGATATTAAAGAACTAGAAAAATCGAATAAAGTGAGAAATACGAACAAAGCAGACCTAATAGCAATGTTGAAAGAAAAAGCAGAAAAAGGTCCAAATATCAATAATAACAACAGTGAACAAACTGAGAATGCGGCTATAAATGAAGAGGCTTCAGGAGCCGACCGACCAGCTATACAAGTGGAGCGTCGTCATCCAGGATTGCCATCGGATAGCGCAGCGGAAATTAAAAAAAGAAGGAAAGCCATAGCATCATCGGATAGTGAGCTTGAAAGCCTTACTTATCCGGATAAACCAACAAAAGTAAATAAGAAAAAAGTGGCGAAAGAGTCAGTTGCGGATGCTTCTGAAAGTGACTTAGATTCTAGCATGCAGTCAGCAGATGAGTCTTCACCACAACCTTTAAAAGCAAACCAACAACCATTTTTCCCTAAAGTATTTAAAAAAATAAAAGATGCGGGGAAATGGGTACGTGATAAAATCGACGAAAATCCTGAAGTAAAGAAAGCGATTGTTGATAAAAGTGCAGGGTTAATTGACCAATTATTAACCAAAAAGAAAAGTGAAGAGGTAAATGCTTCGGACTTCCCGCCACCACCTACGGATGAAGAGTTAAGACTTGCTTTGCCAGAGACACCAATGCTTCTTGGTTTTAATGCTCCTGCTACATCAGAACCGAGCTCATTCGAATTTCCACCACCACCTACGGATGAAGAGTTAAGACTTGCTTTGCCAGAGACGCCAATGCTTCTTGGTTTTAATGCTCCTGCTACATCGGAACCGAGCTCGTTCGAATTTCCACCGCCTCCAACAGAAGATGAACTAGAAATCATCCGGGAAACAGCATCCTCGCTAGATTCTAGTTTTACAAGAGGGGATTTAGCTAGTTTGAGAAATGCTATTAATCGCCATAGTCAAAATTTCTCTGATTTCCCACCAATCCCAACAGAAGAAGAGTTGAACGGGAGAGGCGGTAGACCA

SEQ ID NO: 6 - protein, 32 aa, Listeria monocytogenes
KENSISSMAPPASPPASPKTPIEKKHADEIDK

SEQ ID NO: 7 - protein, 19 aa, Listeria monocytogenes
KENSISSMAPPASPPASPK

SEQ ID NO: 8 - protein, 14 aa, Listeria monocytogenes
KTEEQPSEVNTGPR

SEQ ID NO: 9 - protein, 28 aa, Listeria monocytogenes
KASVTDTSEGDLDSSMQSADESTPQPLK

SEQ ID NO: 10 - protein, 20 aa, Listeria monocytogenes
KNEEVNASDFPPPPTDEELR

SEQ ID NO: 11 - protein, 33 aa, Listeria monocytogenes
RGGIPTSEEFSSLNSGDFTDDENSETTEEEIDR

SEQ ID NO: 12 - protein, 19 aa, Listeria monocytogenes
RSEVTISPAETPESPPATP

SEQ ID NO: 13 - protein, 17 aa, Listeria monocytogenes
KQNTASTETTTTNEQPK

SEQ ID NO: 14 - protein, 17 aa, Listeria monocytogenes
KQNTANTETTTTNEQPK

SEQ ID NO: 15 - protein, 574 aa, Homo sapiens [sequence omitted]

SEQ ID NO: 16 - DNA, 1260 bp, Mus musculus
actgtgacctggtattcagactccctgaacatgagcactgtgaacttccctgccctcggttctgaactcaaggtcaccaccagccaagtgaccagctggggcaagtcagccaagaacttcacatgccacgtgacacatcctccatcattcaacgaaagtaggactatcctagttcgacctgtcaacatcactgagcccaccttggagctactccattcatcctgcgaccccaatgcattccactccaccatccagctgtactgcttcatttatggccacatcctaaatgatgtctctgtcagctggctaatggacgatcgggagataactgatacacttgcacaaactgttctaatcaaggaggaaggcaaactagcctctacctgcagtaaactcaacatcactgagcagcaatggatgtctgaaagcaccttcacctgcaaggtcacctcccaaggcgtagactatttggcccacactcggagatgcccagatcatgagccacggggtgtgattacctacctgatcccacccagccccctggacctgtatcaaaacggtgctcccaagcttacctgtctggtggtggacctggaaagcgagaagaatgtcaatgtgacgtggaaccaagagaagaagacttcagtctcagcatcccagtggtacactaagcaccacaataacgccacaactagtatcacctccatcctgcctgtagttgccaaggactggattgaaggctacggctatcagtgcatagtggaccaccctgattttcccaagcccattgtgcgttccatcaccaagaccccaggccagcgctcagcccccgaggtatatgtgttcccaccaccagaggaggagagcgaggacaaacgcacactcacctgtttgatccagaacttcttccctgaggatatctctgtgcagtggctgggggatggcaaactgatctcaaacagccagcacagtaccacaacacccctgaaatccaatggctccaatcaaggcttcttcatcttcagtcgcctagaggtcgccaagacactctggacacagagaaaacagttcacctgccaagtgatccatgaggcacttcagaaacccaggaaactggagaaaacaatatccacaagccttggtaacacctccctccgtccctcctaggcctccatgtagctgtggtggggaaggtggatgacagacatccgctcactgttgtaacaccaggaagctaccccaataaacactcagtgcctg

SEQ ID NO: 17 - protein, 388 aa, Mus musculus
TVTWYSDSLNMSTVNFPALGSELKVTTSQVTSWGKSAKNFTCHVTHPPSFNESRTILVRPVNITEPTLELLHSSCDPNAFHSTIQLYCFIYGHILNDVSVSWLMDDREITDTLAQTVLIKEEGKLASTCSKLNITEQQWMSESTFTCKVTSQGVDYLAHTRRCPDHEPRGVITYLIPPSPLDLYQNGAPKLTCLVVDLESEKNVNVTWNQEKKTSVSASQWYTKHHNNATTSITSILPVVAKDWIEGYGYQCIVDHPDFPKPIVRSITKTPGQRSAPEVYVFPPPEEESEDKRTLTCLIQNFFPEDISVQWLGDGKLISNSQHSTTTPLKSNGSNQGFFIFSRLEVAKTLWTQRKQFTCQVIHEALQKPRKLEKTISTSLGNTSLRPS

SEQ ID NO: 18 - DNA, 439 bp, Homo sapiens
aggctgatttttgaagaaaggggttgtagcctaaaagatgatggtgttaagtcttctgtacctgttgacagcccttccgggtatcctgtcagaggtgcagcttcaggagtcaggacctagcctcgtgaaaccttctcagactctgtccctcacatgttctgtcactggcgactccatcaccagtggttactggaactggatccggcaagtcccagggaataaacttgagtacatgggtttcataaattacagtggtaacacttactacaatccatctctgagaagtcgaatctccatcactcgagacacatccaagaaccagtacttcctgcacttgaattctgtgactactgaggacacagccacatattactgtgcaagggctaactgggacgtctttgcttactggggccaagggactctggtcactgtctctgca

SEQ ID NO: 19 - protein, 134 aa, Mus musculus
MMVLSLLYLLTALPGILSEVQLQESGPSLVKPSQTLSLTCSVTGDSITSGYWNWIRQVPGNKLEYMGFINYSGNTYYNPSLRSRISITRDTSKNQYFLHLNSVTTEDTATYYCARANWDVFAYWGQGTLVTVSA

SEQ ID NO: 20 - DNA, 22 bp, Unknown
  PCR primer
ggcccgggccccctcctttgat

SEQ ID NO: 21 - DNA, 26 bp, Unknown
  PCR primer
ggtctagatcataatttacttcatcc

SEQ ID NO: 22 - DNA, 26 bp, Unknown
  PCR primer
ggtctagagaattccagcaaaagcag

SEQ ID NO: 23 - DNA, 27 bp, Unknown
  PCR primer
gggtcgacaagggtatttttctttaat

SEQ ID NO: 24 - DNA, 22 bp, Unknown
  PCR primer
ggctcgagcatggagatacacc

SEQ ID NO: 25 - DNA, 28 bp, Unknown
  PCR primer
ggggactagtttatggtttctgagaaca

SEQ ID NO: 26 - DNA, 31 bp, Unknown
  PCR primer
gggggctagccctcctttgattagtatattc

SEQ ID NO: 27 - DNA, 28 bp, Unknown
  PCR primer
ctccctcgagatcataatttacttcatc

SEQ ID NO: 28 - DNA, 55 bp, Unknown
  PCR primer
gactacaaggacgatgaccgacaagtgataacccgggatctaaataaatccgttt

SEQ ID NO: 29 - DNA, 27 bp, Unknown
  PCR primer
cccgtcgaccagctcttcttggtgaag

SEQ ID NO: 30 - DNA, 25 bp, Unknown
  PCR primer
gcggatcccatggagatacacctac

SEQ ID NO: 31 - DNA, 22 bp, Unknown
  PCR primer
gctctagattatggtttctgag

SEQ ID NO: 32 - DNA, 31 bp, Unknown
  PCR primer
ggggtctagacctcctttgattagtatattc

SEQ ID NO: 33 - DNA, 45 bp, Unknown
  PCR primer
atcttcgctatctgtcgccgcggcgcgtgcttcagtttgttgcgc

SEQ ID NO: 34 - DNA, 45 bp, Unknown
  PCR primer
gcgcaacaaactgaagcagcggccgcggcgacagatagcgaagat

SEQ ID NO: 35 - DNA, 42 bp, Unknown
  PCR primer
tgtaggtgtatctccatgctcgagagctaggcgatcaatttc

SEQ ID NO: 36 - DNA, 42 bp, Unknown
  PCR primer
ggaattgatcgcctagctctcgagcatggagatacacctaca

SEQ ID NO: 37 - DNA, 42 bp, Unknown
  PCR primer
aaacggatttatttagatcccgggttatggtttctgagaaca

SEQ ID NO: 38 - DNA, 42 bp, Unknown
  PCR primer
tgttctcagaaaccataacccgggatctaaataaatccgttt

SEQ ID NO: 39 - DNA, 28 bp, Unknown
  PCR primer
gggggtcgaccagctcttcttggtgaag

SEQ ID NO: 40 - protein, 9 aa, Human papillomavirus type 16
RAHYNIVTF

SEQ ID NO: 41 - protein, 9 aa, Listeria monocytogenes
GYKDGNEYI

SEQ ID NO: 42 - protein, 9 aa, Mus musculus
LYCFIYGHI

SEQ ID NO: 43 - protein, 9 aa, Mus musculus
IYGHILNDV

SEQ ID NO: 44 - DNA, 2048 bp, Listeria monocytogenes
taacgacgataaagggacagcaggactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatcgttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatcctgaaggtaacgaaattgttcaacataaaaactggagcgaaaacaataaaagcaagctagctcatttcacatcgtccatctatttgccaggtaacgcgagaaatattaatgtttacgctaaagaatgcactggtttagcttgggaatggtggagaacggtaattgatgaccggaacttaccacttgtgaaaaatagaaatatctccatctggggcaccacgctttatccgaaatatagtaataaagtagataatccaatcgaataattgtaaaagtaataaaaaattaagaataaaaccgcttaacacacacgaaaaaataagcttgttttgcactcttcgtaaattattttgtgaagaatgtagaaacaggcttattttttaatttttttagaagaattaacaaatgtaaaagaatatctgactgtttatccatataatataagcatatcccaaagtttaagccacctatagtttctactgcaaaacgtataatttagttcccacatatactaaaaaacgtgtccttaactctctctgtcagattagttgta